SEQUENCE LISTING (86 sequences)

SEQ ID NO: 1 - protein, 183 aa, human
MQAPRAALVFALVIALVPVGRGNYEELENSGDTTVESERPNKVTIPSTFAAVTIKETLNANINSTNFAPDENQLEFILMVLIPLILLVLLLLSVVFLATYYKRKRTKQEPSSQGSQSALQTYELGSENVKVPIFEEDTPSVMEIEMEELDKWMNSMNRNADFECLPTLKEEKESNHNPSDSES

SEQ ID NO: 2 - DNA, 675 bp, human
aagcttagcccggcgcagcatcctgagcgcgcctctgccgaggcgagcggacatgcaggctccccgcgcagccctagtcttcgccctggtgatcgcgctcgttcccgtcggccggggtaattatgaggaattagaaaactcaggagatacaactgtggaatctgaaagaccaaataaagtgactattccaagcacatttgctgcagtgaccatcaaagaaacattaaatgcaaatataaattctaccaactttgctccggatgaaaatcagttagagtttatactgatggtgttaatcccattgattttattggtcctcttacttttatccgtggtattccttgcaacatactataaaagaaaaagaactaacaagaaccttctagccaaggatctcagagtgctttacagacatatgaactgggaagtgaaaacgtgaaagtccctatttttgaggaagatacaccctctgttatggaaattgaaatggaagagcttgataaatggatgaacagcatgaatagaaatgccgactttgaatgtttacctaccttgaaggaagagaaggaatcaaatcacaacccaagtgacagtgaatcctaaacctgaatggcgctcatgttttccaagagaagcagcccctgagggagtctgctgaggctgccaacaggatcc

SEQ ID NO: 3 - protein, 181 aa, murine
MTVPCAALVLALGLAFGQSSQGNDEESEYSGQSITEEENSEDETTRSALATVTTEALAENVNSTHTNDTSNQVEFILMVAIPLAALLILLFMVLIATYFKSKRPKQEPSSQGSQSALQTHELGGETLKVPIFEEDTPSVMEIEMEELDKWMNSMNRNADYECLPTLKEEKEPNPSPSDNES

SEQ ID NO: 4 - protein, 367 aa, rat
MTRPPYQEAPVGDLQMGDRQESSGDKDRNDEDSEYSGHSTTEEDTAEEETTRALATVTTEALAESANSTHIHGTSNQVEFILMVAVPLAALLILLFAILIVIYFKSRRPKQEPSSQGSQSALQTLRLLLSLETKRPEPSVAPSLGPRPTIPLPTAQRGPCQQSGCKAGTKGGRQDRGENEMAGRKGTKWKPVGNGPGAEKMRPQKAFCSFNADYGASHSVHLEHFGNGFLNFSIICMQVGFCPPPSLWGAQMRVEIRAHSGTVEPLAVWEIGGEVAKQGKGTDDLGGETLKVPIFEEDTPSVMEIEMEELDKWMNSMNRNGTWKTKAFACLCGNAGLDGCLCFISNSENLKLCFIWHSTCALLKDPV

SEQ ID NO: 5 - DNA, 703 bp, artificial sequence
  FLJ32028 with an HA epitope tag
aagcttagcccggcgcagcatcctgagcgcgcctctgccgaggcgagcggacatgcaggctccccgcgcagccctagtcttcgccctggtgatcgcgctcgttcccgtcggccggggtaattatccatatgatgttccagattatgcttatgaggaattagaaaactcaggagatacaactgtggaatctgaaagaccaaataaagtgactattccaagcacatttgctgcagtgaccatcaaagaaacattaaatgcaaatataaattctaccaactttgctccggatgaaaatcagttagagtttatactgatggtgttaatcccattgattttattggtcctcttacttttatccgtggtattccttgcaacatactataaaagaaaaagaactaaacaagaaccttctagccaaggatctcagagtgctttacagacatatgaactgggaagtgaaaacgtgaaagtccctatttttgaggaagatacaccctctgttatggaaattgaaatggaagagcttgataaatggatgaacagcatgaatagaaatgccgactttgaatgtttacctaccttgaaggaagagaaggaatcaaatcacaacccaagtgacagtgaatcctaaacctgaatggcgctcatgttttccaagagaagcagcccctgagggagtctgctgaggctgccaacaggatcc

SEQ ID NO: 6 - protein, 192 aa, artificial sequence
  FLJ32028 with HA epitope tag
MQAPRAALVFALVIALVPVGRGNYPYDVPDYAYEELENSGDTTVESERPNKVTIPSTFAAVTIKETLNANINSTNFAPDENQLEFILMVLIPLILLVLLLLSVVFLATYYKRKRTKQEPSSQGSQSALQTYELGSENVKVPIFEEDTPSVMEIEMEELDKWMNSMNRNADFECLPTLKEEKESNHNPSDSES

SEQ ID NO: 7 - DNA, 637 bp, artificial sequence
  FLJ32028 with HA epitope tag
aagcttagcccggcgcagcatcctgagcgcgcctctgccgaggcgagcggacatgcaggctccccgcgcagccctagtcttcgccctggtgatcgcgctcgttcccgtcggccggggtaattatgaggaattagaaaactcaggagatacaactgtggaatctgaaagaccaaataaagtgactattccaagcacatttgctgcagtgaccatcaaagaaacattaaatgcaaatataaattctaccaactttgctccggatgaaaatcagttagagtttatactgatggtgttaatcccattgattttattggtcctcttacttttatccgtggtattccttgcaacatactataaaagaaaaagaactaaacaagaaccttctagccaaggatctcagagtgctttacagacatatgaactgggaagtgaaaacgtgaaagtccctatttttgaggaagatacaccctctgttatggaaattgaaatggaagagcttgataaatggatgaacagcatgaatagaaatgccgactttgaatgtttacctaccttgaaggaagagaaggaatcaaatcacaacccaagtgacagtgaatcctatccatatgatgttccagattatgcttaaggatcc

SEQ ID NO: 8 - protein, 192 aa, artificial sequence
  FLJ32028 with HA epitope tag
MQAPRAALVFALVIALVPVGRGNYEELENSGDTTVESERPNKVTIPSTFAAVTIKETLNANINSTNFAPDENQLEFILMVLIPLILLVLLLLSVVFLATYYKRKRTKQEPSSQGSQSALQTYELGSENVKVPIFEEDTPSVMEIEMEELDKWMNSMNRNADFECLPTLKEEKESNHNPSDSESYPYDVPDYA

SEQ ID NO: 9 - DNA, 1421 bp, murine
  misc_feature (40)..(40): n = degenefacy in code
atgttttgatgacccaaactccactctccctgcctgtccnttttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaacgggctgatgctgcaccaactgtatccatcttcccaccatccagtgagcagttaacatccggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaagcggccgcactagatataattaaggagataaatatgaaatatctgctgccgaccgcggcggcgggcctgctgctgctggcggcgcagccggcgatggcgctcgaggtgaagctggtggagtctgggggaggcttagtgaagcctggagggtccctgaaactctcctgtgcagcctctggattcactttcagtgactatgccatgtcttgggttcgccagactccagagaagaggctggagtgggtcgcatcaattagtagtggtggtaccacctattatctagacagtgtgaagggccgattcaccatctccagagataatgccaggaacatcctgtacctgcaaatgagcagtctgaggtctgaggacacggccatgtattattgtgtaagaagtgagacgaactactggggccaaggcaccactctcacagtctcctcagccaaaacgacacccccatctgtctatccactggcccctggatctgctgcccaaactaactccatgataaccctaggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccatccagcagcttgggcacccagacctacatctgcaacgtgaatcacaagcccagcaacaccaaggtggacaagaaagttgagcccaaatcttgtgacaaaactagt

SEQ ID NO: 10 - protein, 474 aa, murine
  MISC_FEATURE (14)..(14): Xaa = any amino acid
YVLMTQTPLSLPVXFGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECXAAALDIIKEINMKYLLPTAAAGLLLLAAQPAMALEVKLVESGGGLVKPGGSLKLSCAASGFTFSDYAMSWVRQTPEKRLEWVASISSGGTTYYLDSVKGRFTISRDNARNILYLQMSSLRSEDTAMYYCVRSETNYWGQGTTLTVSSAKTTPPSVYPLAPGSAAQTNSMITLGCLVKDYFPEPVTVSWNSGALTSGVHTFPAVLQSSGLYSLSSVVTVPSSSLGTQTYICNVNHKPSNTKVDKKVEPKSCDKTS

SEQ ID NO: 11 - DNA, 1421 bp, murine
agacattgtggatgactcaggctgaactctccagtcctgtcacttctggagaatcagtttccatctcctgcaggtctagtaagagtctcctatataaggatgggaagacatacttgaattggtatctgcagagaccaggacaatctcctcagctcctgatctattttatgtccacccgtgcaccaggagtctcagaccggtttagtggcattgggtcaggaacagatttcaccctggaaatcagtagagtgaaggctgaggatgtgggtgtgtattattgtcaacaacttgtagagtatcctctcacgttcggtgctgggaccaagctggaactgaaacgggctgatgctgcaccaactgtatccatcttcccaccatccagtgagcagttaacatccggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaagcggccgcactagatataattaaggagataaatatgaaatatctgctgccgaccgcggcggcgggcctgctgctgctggcggcgcagccggcgatggcgctcgaggtccaactgcagcagtctggggctgagctggtgaggcctggggcttcagtgacgctgtcctgcaaggcctcggactacacattcactgactatgaaatgcactgggtgaagcagacacctgtgcatggcctggaatggattggaggtattgatcctgaaactggtggtactgtctacaatcagaagctcaagggcaaggccacactgactgcagacaaagcctccagcacggcctacatggagctccgaagcctgacatctgaggactctgccgtctattactgtacggctggtgtttattggggccaagggactctggtcactgtctctgcagccaaaacaacagccccatcggtctatccactggcccctgtgtgtggagatacaactggctcctcgatgaccctaggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccatccagcagcttgggcacccagacctacatctgcaacgtgaatcacaagcccagcaacaccaaggtggacaagaaagttgagcccaaatcttgtgacaaaactagt

SEQ ID NO: 12 - protein, 472 aa, murine
TLWMTQAELSSPVTSGESVSISCRSSKSLLYKDGKTYLNWYLQRPGQSPQLLIYFMSTRAPGVSDRFSGIGSGTDFILEISRVKAEDVGVYYCQQLVEYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECAAALDIIKEINMKYLLPTAAAGLLLLAAQPAMALEVQLQQSGAELVRPGASVTLSCKASDYTFTDYEMHWVKQTPVHGLEWIGGIDPETGGTVYNQKLKGKATLTADKASSTAYMELRSLTSEDSAVYYCTAGVYWGQGTLVTVSAAKTTAPSVYPLAPVCGDTTGSSMTLGCLVKDYFPEPVTVSWNSGALTSGVHTFPAVLQSSGLYSLSSVVTVPSSSLGTQTYICNVNHKPSNTKVDKKVEPKSCDKTS

SEQ ID NO: 13 - protein, 108 aa, murine
DIQMTQTTSSLSASLGDRVTISCRTSQDISNYLNWYQQKPDGTVKVLIYYTSRLHSGVPSRFSGSGSGTDYSLTINNLEQEDIATYFCQQGNTLPFTFGSGTKLEIKR

SEQ ID NO: 14 - protein, 113 aa, murine
DIVMTQAELSSPVTSGESVSISCRSSKSLLYKDGKTYLNWYLQRPGQSPQLLIYFMSTRAPGVSDRFSGIGSGTDFTLEISRVKAEDVGVYYCQQLVEYPLTFGAGTKLELKR

SEQ ID NO: 15 - protein, 114 aa, murine
DIVMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELKR

SEQ ID NO: 16 - protein, 114 aa, murine
DIVMSQSPSSLAVSVGEKVTMSCKSSQSLLYSSNQKNYLAWYQQKPGQSPKLLIYWASARGSGVPDRFTGSGSGTDFTLTISSVKAEDLAVYYCQQYYSYPLTIGAGTKLELKR

SEQ ID NO: 17 - protein, 113 aa, murine
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKR

SEQ ID NO: 18 - protein, 113 aa, murine
LEVQLQQSGAELVRPGASVTLSCKASGYTFTDYEMHWVKQTPVHGLEWIGGIDPEIGGTVYNQKFKGKATLTADKSSGTAYMELRSLTSEDSAVYYCTSFAYWGQGTLVTVSA

SEQ ID NO: 19 - protein, 113 aa, murine
LEVQLQQSGAELVRPGASVTLSCKASDYTFTDYEMHWVKQTPVHGLEWIGGIDPETGGTVYNQKLKGKATLTADKASSTAYMELRSLTSEDSAVYYCTAGVYWGQGTLVTVSA

SEQ ID NO: 20 - protein, 113 aa, murine
LEVQLQQSGAELVRPGASVTLSCKASGYTFTDYEVHWVKQTPVQGLDWIGGIDPESGGTAYNQKFKGKATLTADKSSRTAYMELRSLTSEDSAVYYCTAGADWGQGTLVTVFA

SEQ ID NO: 21 - protein, 116 aa, murine
LEVQLKQSGAELVKPGASVKLSCTASGFNIKDTYINWVKQRPEQGLEWIGRIDPANNNTNYDPKFQGKATITADTPSNTAYLQLSSLTSEDTDVYYCVSGGYFDYWGQGTTLTVSS

SEQ ID NO: 22 - protein, 116 aa, murine
LEVQLQQSGAEFVRPGASVKLSCTGSGFNIKDTYMNWVIQRPEQGLEWIGMIDPANGNTQYDPKFQGKATITADTSSNTAYLQLSSLTSEDTAVYYCTSGGYFDYWGQGTTLTVSS

SEQ ID NO: 23 - protein, 114 aa, murine
LEVKLVESGGGLVKPGGSLKLSCAASGFTFSDYAMSWVRQTPEKRLEWVASISSGGTTYYLDSVKGRFTISRDNARNILYLQMSSLRSEDTAMYYCVRSETNYWGQGTTLTVSS

SEQ ID NO: 24 - protein, 120 aa, murine
LEVQLVESGGGLVQPKGSLKLSCAASGFNFNTYAMNWVRQSPGKGLEWVARIRTKSNNYATYYADSVKDRFSVSRDDSQSMLYLQMNNLKTEDTAMYYCVRHEGDWFAYWGQGTLVTVSE

SEQ ID NO: 25 - protein, 120 aa, murine
LEVQLVESGGGLVQPKGSLKLSCAASGFNFNTYAMNWVRQAPGKGLEWVARIRSKSNNYATYYADSVKDRFTISRDDSQSMLYLQMNNLKTEDTAMYYCVRHEGDWFAYWGQGTLVTVSA

SEQ ID NO: 26 - protein, 120 aa, murine
LEVKLVESGGGLVQPKGSLKLSCAASGFNFNTYAMNWVRQAPGKGLEWVARIRSKSNNYATYYADSVKDRFTISRDDSQSMLYLQMNNLKTEDTAMYYCVRHEGDWFAYWGQGTLVTVSA

SEQ ID NO: 27 - protein, 120 aa, murine
LEVQLVESGGGLVQPKGSLKLSCAASGFNFNTYAMNWVRQAPGKGLEWVARIRSKSNNYATYYADSVKDRFTISRDDSQSMLYLQMNNLKTEDTAMYYCVRHEGNWFAYWGQGTLVTVSA

SEQ ID NO: 28 - protein, 116 aa, murine
LEVQLQQSGAELAKPGASVKMSCKASGYTFTNSWIHWVKQRPGQGLEWIGYIHPGPGYTEYNQNFKDKATLTADKSSSTAYIQLSSLTSEDSAVYYCIRGGDWGYWGQGTSVTVSS

SEQ ID NO: 29 - protein, 116 aa, murine
LEVQLKQSGAELVKPGASVKLSCTASGFNIKDTYMNWVKQRPEQGLEWIGGIDPANDNTEYVPKFQGRATITADTSSNTAYLQLRSLTSDDTAVYYCVTGGYFDYWGQGTTLTVSS

SEQ ID NO: 30 - protein, 116 aa, murine
LEVQLQQSGAELVKPGASVKLSCTASGFNIKDTYMNWVKQRPEQGLEWIGGIDPANDNTEYVPKFQGRATITADTSSNTAYLQLRSLTSDDTAVYYCVTGGYFDYWGQGTTLTVSS

SEQ ID NO: 31 - protein, 113 aa, murine
LEVQLQQSGAELVRPGASVTLSCKASGYTFTDYEMHWVKQTPVHGLEWIGGIDPETGGTVYNQKFKGKATLTADKSSSTAYMELRSQTSEDSAVYYCTRWDYWGQGTTLTVSS

SEQ ID NO: 32 - protein, 120 aa, murine
LEVQLVESGGGLVQPKGSLKLSCAASGFTFNTYAMNWVRQAPGKGLEWVARIRTKSNNYATYYADSVKDRFTISRDDSQSMLYLQMNNLKTEDTATYYCVRQGENRFAYWGQGTLVTVSA

SEQ ID NO: 33 - protein, 113 aa, murine
LEVQLQQSGAELVRPGASVTLSCKASGYTFTDYEMHWVKQTHVHGLEWIGGIDPETGGTVYNQKFKGKATLTADKSSSTAYMELRSLTSEDSAVYYCTSSLPWGQGTLVTVSA

SEQ ID NO: 34 - protein, 6 aa, murine
QDISNY

SEQ ID NO: 35 - protein, 11 aa, murine
KSLLYKDGKTY

SEQ ID NO: 36 - protein, 12 aa, murine
QSLLYSSNQKNY

SEQ ID NO: 37 - protein, 11 aa, murine
QSIVHSNGNTY

SEQ ID NO: 38 - protein, 10 aa, murine
GYTFTDYEMH

SEQ ID NO: 39 - protein, 10 aa, murine
DYTFTDYEMH

SEQ ID NO: 40 - protein, 10 aa, murine
GYTFTDYEVH

SEQ ID NO: 41 - protein, 10 aa, murine
GFNIKDTYIN

SEQ ID NO: 42 - protein, 10 aa, murine
GFTFSDYAMS

SEQ ID NO: 43 - protein, 10 aa, murine
GFNFNTYAMN

SEQ ID NO: 44 - protein, 10 aa, artificial sequence
  primer
GYTFTNSWIH

SEQ ID NO: 45 - protein, 10 aa, murine
GFNIKDTYMN

SEQ ID NO: 46 - protein, 10 aa, murine
GYTFTDYEMH

SEQ ID NO: 47 - protein, 10 aa, murine
GFTFNTYAMN

SEQ ID NO: 48 - protein, 10 aa, murine
GYTFTDYEMH

SEQ ID NO: 49 - protein, 3 aa, murine
YTS

SEQ ID NO: 50 - protein, 3 aa, murine
FMS

SEQ ID NO: 51 - protein, 3 aa, murine
WAS

SEQ ID NO: 52 - protein, 3 aa, murine
KVS

SEQ ID NO: 53 - protein, 17 aa, murine
GIDPEIGGTVYNQKFKG

SEQ ID NO: 54 - protein, 17 aa, murine
GIDPETGGTVYNQKLKG

SEQ ID NO: 55 - protein, 17 aa, murine
GIDPESGGTAYNQKFKG

SEQ ID NO: 56 - protein, 17 aa, murine
RIDPANNNTNYDPKFQG

SEQ ID NO: 57 - protein, 17 aa, murine
MIDPANGNTQYDPKFQG

SEQ ID NO: 58 - protein, 16 aa, murine
SISSGGTTYYLDSVKG

SEQ ID NO: 59 - protein, 19 aa, murine
RIRTKSNNYATYYADSVKD

SEQ ID NO: 60 - protein, 19 aa, murine
RIRSKSNNYATYYADSVKD

SEQ ID NO: 61 - protein, 17 aa, murine
YIHPGPGYTEYNQNFKD

SEQ ID NO: 62 - protein, 17 aa, murine
GIDPANDNTEYVPKFQG

SEQ ID NO: 63 - protein, 17 aa, murine
GIDPETGGTVYNQKFKG

SEQ ID NO: 64 - protein, 19 aa, murine
RIRTKSNNYATYYADSVKD

SEQ ID NO: 65 - protein, 17 aa, murine
GIDPETGGTVYNQKFKG

SEQ ID NO: 66 - protein, 13 aa, murine
QQGNTLPFTFGSG

SEQ ID NO: 67 - protein, 13 aa, murine
QQLVEYPLTFGAG

SEQ ID NO: 68 - protein, 13 aa, murine
QQYYSYPLTFGAG

SEQ ID NO: 69 - protein, 13 aa, murine
QQYYSYPLTIGAG

SEQ ID NO: 70 - protein, 13 aa, murine
FQGSHVPLTFGAG

SEQ ID NO: 71 - protein, 3 aa, murine
FAY

SEQ ID NO: 72 - protein, 3 aa, murine
GVY

SEQ ID NO: 73 - protein, 3 aa, murine
GAD

SEQ ID NO: 74 - protein, 6 aa, murine
GGYFDY

SEQ ID NO: 75 - protein, 5 aa, murine
SETNY

SEQ ID NO: 76 - protein, 8 aa, murine
HEGDWFAY

SEQ ID NO: 77 - protein, 8 aa, murine
HEGNWFAY

SEQ ID NO: 78 - protein, 6 aa, murine
GGDWGY

SEQ ID NO: 79 - protein, 6 aa, murine
GGYFDY

SEQ ID NO: 80 - protein, 3 aa, murine
WDY

SEQ ID NO: 81 - protein, 8 aa, murine
QGENRFAY

SEQ ID NO: 82 - protein, 3 aa, murine
SLP

SEQ ID NO: 83 - DNA, 663 bp, human
agcccggcgcagcatcctgagcgcgcctctgccgaggcgagcggacatgcaggctccccgcgcagccctagtcttcgccctggtgatcgcgctcgttcccgtcggccggggtaattatgaggaattagaaaactcaggagatacaactgtggaatctgaaagaccaaataaagtgactattccaagcacatttgctgcagtgaccatcaaagaaacattaaatgcaaatataaattctaccaactttgctccggatgaaaatcagttagagtttatactgatggtgttaatcccattgattttattggtcctcttacttttatccgtggtattccttgcaacatactataaaagaaaaagaactaacaagaaccttctagccaaggatctcagagtgctttacagacatatgaactgggaagtgaaaacgtgaaagtccctatttttgaggaagatacaccctctgttatggaaattgaaatggaagagcttgataaatggatgaacagcatgaatagaaatgccgactttgaatgtttacctaccttgaaggaagagaaggaatcaaatcacaacccaagtgacagtgaatcctaaacctgaatggcgctcatgttttccaagagaagcagcccctgagggagtctgctgaggctgccaaca

SEQ ID NO: 84 - protein, 182 aa, human
MQAPRAALVFALVIALVPVGRGNYEELENSGDTTVESERPNKVTIPSTFAAVTIKTLNANINSTNFAPDENQLEFILMVLIPLILLVLLLLSVVFLATYYKRKRTKQEPSSQGSQSALQTYELGSENVKVPIFEEDTPSVMEIEMEELDKWMNSMNRNADFECLPTLKEEKESNHNPSDSES

SEQ ID NO: 85 - protein, 181 aa, artificial sequence
  Majority Sequence of human FLJ32028 and similar, murine and rat proteins
MTXPXAALVXALXXAXXQXSXGNDEESEYSGXSTTEEEXXEXETTRSALATVTTEALAENXNSTHXXXTSNQVEFILMVAIPLAALLILLFXVLIATYFKSKRPKQEPSSQGSQSALQTXELGGETLKVPIFEEDTPSVMEIEMEELDKWMNSMNRNADFECLPTLKEEKEXNXXPSDSES

SEQ ID NO: 86 - protein, 120 aa, murine; identical to SEQ ID NO: 24

(19) United States
(12) Patent Application Publication
(10) Pub. No.: US 2008/0057519 A1
(43) Pub. Date: Mar. 6, 2008

(54) Cell Surface Protein Associated with Human Chronic Lymphocytic Leukemia

(76) Inventor: John McWhirter, Chula Vista, CA (US)

Correspondence Address: ROPES & GRAY LLP;PATENT DOCKETING 39/361, 1211 AVENUE OF THE AMERICAS, NEW YORK, NY, 10036-8704, US

(21) Appl. No.: 10/559,438

(22) PCT Filed: Jun. 2, 2004
(86) PCT No.: PCT/US04/17118

§ 371 Date: Aug. 21, 2007

Related U.S. Application Data
(60) Provisional application No. 60/475,156, filed on Jun. 2, 2003.
(60) Provisional application No. 60/530,094, filed on Dec. 15, 2003.

(51) Int. Cl. C07K 16/18; C12N 15/00; C12N 15/11; C12N 5/06; C12P 21/04; G01N 33/574

(52) U.S. Cl. 435/7.23; 435/320.1; 435/325; 435/69.1; 530/387.3; 530/387.9; 530/388.1; 536/23.1

(57) ABSTRACT

Protein (referred to herein as “FLJ32028”) that is associated with B-cell chronic lymphocytic leukemia is isolated. Isolated nucleic acid encoding the protein, the generation of monoclonal antibodies recognizing at least a portion of this protein, and the use of this protein or antibodies thereto as a diagnostic marker or therapeutic target for B-CLL are also disclosed.

### RELATED APPLICATIONS

This application claims priority to U.S. Provisional Application Nos. 60/475,156 and 60/530,094, filed Jun. 2, 2003 and Dec. 15, 2003, respectively, the entire disclosures of which are incorporated herein by this reference.

### BACKGROUND

1. Technical Field

The present disclosure relates to an isolated protein (referred to herein as “FLJ32028”) that is associated with B-cell chronic lymphocytic leukemia. Isolated nucleic acid encoding the protein, the generation of monoclonal antibodies recognizing at least a portion of this protein, and the use of this protein or antibodies thereto as a diagnostic marker or therapeutic target for B-CLL are also disclosed.

2. Background of Related Art

B cell chronic lymphocytic leukemia (“B-CLL”) is a disease of the white blood cells. It is the most common form of leukemia in the Western Hemisphere. The hallmark of B-CLL is the growth of malignant CD5⁺ B lymphocytes that grow slowly but have an extended life span.

B-CLL accounts for approximately 30% of all leukemias. Although it occurs more frequently in individuals over 50 years of age, it is increasingly seen in younger people. B-CLL is characterized by the clonal expansion of mature B cells with a B1-like phenotype (CD5⁺, CD19⁺, CD23⁺, CD79b⁻sIg^{low}). These cells are defective for normal B cell receptor (“BCR”) signaling and are therefore non-functional and anergic. B lymphocytes normally function to fight infection but in B-CLL they accumulate in the blood, bone marrow, and lymph nodes. The production of normal bone marrow and blood cells is impaired and patients often experience severe anemia as well as low platelet counts. This places them at risk for life-threatening bleeding and the development of severe infections due to reduced numbers of white blood cells.

It has been shown that chronic lymphocytic leukemia (“CLL”) patients can be divided into two groups based on prognosis. Roughly 50% of patients have malignant cells with somatically mutated VH genes. These patients generally have a good prognosis (median survival of 26 years) and do not require any treatment. The other 50% have malignant cells with unmutated, germline VH genes. These patients generally have a poor prognosis (median survival of 8 years) and require immediate treatment. Other unfavorable prognostic indicators associated with the second group are the presence of a high percentage of CD38+ cells, the presence of mutant p53, and a gene expression profile similar to activated B cells.

Recently there has been some success in treating high-risk B-CLL patients with monoclonal antibody therapy. One drug, Alemtuzamab (Campath or Anti-CD52), has been approved for patients that have failed to respond to chemotherapy and a second drug, Rituximab (Anti-CD20) has shown some efficacy in clinical trials. However, there remains a need to discover cell surface markers associated with B-CLL that have higher expression levels and/or greater specificity. Monoclonal antibodies to such markers may have greater efficacy and fewer side effects than existing therapeutic antibodies.

### SUMMARY

A cDNA clone has been identified that encodes a novel polypeptide believed to be a cell surface protein associated with B-cell chronic lymphocytic leukemia, designated in the present application as “FLJ32028.”

In one embodiment, an isolated nucleic acid molecule comprising DNA encoding a FLJ32028 polypeptide is provided.

In one aspect, the isolated nucleic acid comprises DNA having at least about 80% sequence identity, preferably at least about 85% sequence identity, more preferably at least about 90% sequence identity, most preferably at least about 95% sequence identity to (a) a DNA molecule encoding a FLJ32028 polypeptide having the sequence of amino acid residues from 21 to about 183, inclusive of FIG. 1 (SEQ ID NO: 1), or (b) the complement of the DNA molecule of (a).

In another aspect, the present disclosure concerns an isolated nucleic acid molecule encoding a FLJ32028 polypeptide comprising DNA hybridizing to the complement of the nucleic acid between about residues 50 and about 280, inclusive, of FIG. 1 (SEQ ID NO: 2). Preferably, hybridization occurs under stringent hybridization and wash conditions.

In a further aspect, the present disclosure concerns an isolated nucleic acid molecule comprising DNA having at least about 80% sequence identity, preferably at least about 85% sequence identity, more preferably at least about 90% sequence identity, most preferably at least about 95% sequence identity to (a) a DNA molecule encoding the same mature polypeptide encoded by the human protein FLJ32028 cDNA, or (b) the complement of the DNA molecule of (a). In a particularly useful embodiment, the nucleic acid comprises a DNA encoding the same mature polypeptide encoded by the human protein FLJ32028 cDNA.

In a still further aspect, the present disclosure concerns an isolated nucleic acid molecule comprising (a) DNA encoding a polypeptide having at least about 80% sequence identity, preferably at least about 85% sequence identity, more preferably at least about 90% sequence identity, most preferably at least about 95% sequence identity to the sequence of amino acid residues from about 21 to about 183, inclusive of FIG. 1 (SEQ ID NO: 1), or the complement of the DNA of (a).

In a further aspect, the present disclosure concerns an isolated nucleic acid molecule having at least about 50 nucleotides, and preferably at least about 100 nucleotides and produced by hybridizing a test DNA molecule under stringent conditions with (a) a DNA molecule encoding a FLJ32028 polypeptide having the sequence of amino acid residues from about 21 to about 183, inclusive of FIG. 1 (SEQ ID NO: 1), or (b) the complement of the DNA molecule of (a), and, if the DNA molecule has at least about an 80% sequence identity, preferably at least about an 85% sequence identity, more preferably at least about a 90% sequence identity, most preferably at least about a 95% sequence identity to (a) or (b), isolating the test DNA molecule.

In a specific aspect, the present disclosure provides an isolated nucleic acid molecule comprising DNA encoding a FLJ32028 polypeptide, with or without the N-terminal signal sequence and/or the initiating methionine, and its soluble, i.e. transmembrane domain deleted or inactivated variants, or is complementary to such encoding nucleic acid molecule. The signal peptide has been tentatively identified as extending from about amino acid position 1 through about amino acid position 20 in the sequence of FIG. 1 (SEQ ID NO: 1). The transmembrane domain has been tentatively identified as at about amino acid positions 75 through 100 in the FLJ32028 amino acid sequence (FIG. 1, SEQ ID NO: 1).

In another aspect, the present disclosure concerns an isolated nucleic acid molecule comprising (a) DNA encoding a polypeptide scoring at least about 80% positives, preferably at least about 85% positives, more preferably at least about 90% positives, most preferably at least about 95% positives when compared with the amino acid sequence of residues 21 to about 183, inclusive of (FIG. 1, SEQ ID NO: 1), or (b) the complement of the DNA of (a).

Another embodiment is directed to fragments of a FLJ32028 polypeptide coding sequence that may find use as hybridization probes. Such nucleic acid fragments are from about 20 to about 80 nucleotides in length, preferably from about 20 to about 60 nucleotides in length, more preferably from about 20 to about 50 nucleotides in length, and most preferably from about 20 to about 40 nucleotides in length.

In another embodiment, the present disclosure provides isolated FLJ32028 polypeptide encoded by any of the isolated nucleic acid sequences hereinabove defined.

In a specific aspect, the present disclosure provides isolated native sequence FLJ32028 polypeptide, which in one embodiment, includes an amino acid sequence comprising residues 21 through 183 of (FIG. 1, SEQ ID NO: 1).

In another aspect, the present disclosure concerns an isolated FLJ32028 polypeptide, comprising an amino acid sequence having at least about 80% sequence identity, preferably at least about 85% sequence identity, more preferably at least about 90% sequence identity, most preferably at least about 95% sequence identity to the sequence of amino acid residues 21 to about 183, inclusive of (FIG. 1, SEQ ID NO: 1).

In a further aspect, the present disclosure concerns an isolated FLJ32028 polypeptide, comprising an amino acid sequence scoring at least about 80% positives, preferably at least about 85% positives, more preferably at least about 90% positives, most preferably at least about 95% positives when compared with the amino acid sequence of residues 21 through 183 of(FIG. 1, SEQ ID NO: 1).

In yet another aspect, the present disclosure concerns an isolated FLJ32028 polypeptide, comprising the sequence of amino acid residues 21 to about 183, inclusive of (FIG. 1, SEQ ID NO: 1), or a fragment thereof sufficient to provide a binding site for an anti-FLJ32028 antibody. Preferably, the FLJ32028 fragment retains a qualitative biological activity of a native FLJ32028 polypeptide.

In a still further aspect, the present disclosure provides a polypeptide produced by (i) hybridizing a test DNA molecule under stringent conditions with (a) a DNA molecule encoding a FLJ32028 polypeptide having the sequence of amino acid residues from about 21 to about 183, inclusive of (FIG. 1, SEQ ID NO: 1), or (b) the complement of the DNA molecule of (a), and if the test DNA molecule has at least about an 80% sequence identity, preferably at least about an 85% sequence identity, more preferably at least about a 90% sequence identity, most preferably at least about a 95% sequence identity to (a) or (b), (ii) culturing a host cell comprising the test DNA molecule under conditions suitable for expression of the polypeptide, and (iii) recovering the polypeptide from the cell culture.

In yet another embodiment, the present disclosure concerns agonists and antagonists of a native FLJ32028 polypeptide. In a particular embodiment, the agonist or antagonist is an anti-FLJ32028 antibody.

In a further embodiment, the present disclosure concerns a method of identifying agonists or antagonists of a native FLJ32028 polypeptide, by contacting the native FLJ32028 polypeptide with a candidate molecule and monitoring a biological activity mediated by said polypeptide.

In a still further embodiment, the present disclosure concerns a composition comprising a FLJ32028 polypeptide, or an agonist or antagonist as hereinabove defined, in combination with a pharmaceutically acceptable carrier.

### BRIEF DESCRIPTION OF THE DRAWINGS

FIG. 1 shows the cDNA sequence (SEQ. ID No. 2) and predicted open reading frame of the FLJ32028 gene. Primers F1 and R1 were used to clone the cDNA from primary CLL cells by RT-PCR. The HindIII and BamHI restriction enzyme sites in the primers were used to clone the PCR product into a mammalian expression vector. Predicted features of the protein sequence (SEQ. ID No. 1) are indicated: the signal peptide and transmembrane region are indicated with open black arrows, potential O-linked (G) and N-linked (NG) glycosylation sites are indicated, potential serine/threonine phosphorylation sites (P) are indicated with open red boxes.

FIG. 2 shows the amino acid sequence alignment of the human FLJ32028 protein with similar proteins from mouse (Genbank accession number NP_{—}796234, SEQ. ID No. 3) and rat (Genbank accession number XP_{—}227319, SEQ. ID No. 4). The alignment was created using the Clustal W program. The predicted signal peptide and transmembrane region of the human protein are indicated with black line boxes.

FIG. 3 shows the DNA sequence (SEQ. ID No. 5) and conceptual translation of the FLJ32028 cDNA with an HA epitope tag inserted at the predicted N-terminal end of the protein (SEQ. ID No. 6). The oligonucleotide primers used to construct the cDNA by overlap extension PCR are indicated by solid arrows. The predicted signal peptide, HA tag, and transmembrane region are indicated with open black arrows.

FIG. 4 shows the DNA sequence (SEQ. ID No. 7) and conceptual translation of the FLJ32028 cDNA with an HA epitope tag fused to the predicted C-terminal end of the protein (SEQ. ID No. 8). The oligonucleotide primers used to construct the cDNA by PCR are indicated by solid arrows. The predicted signal peptide, transmembrane region, and HA tag are indicated with open black arrows.

FIG. 5 shows the results of flow cytometric analysis of 293-EBNA cells transiently transfected with the HA epitope-tagged FLJ32028 cDNAs. 293-EBNA cells were cotransfected with the HA-tagged FLJ32028 cDNAs in vector pCEP4 (Invitrogen) and the pEGFP plasmid (Clontech) which expresses the green fluorescent protein (“GFP”). After 48 hours of transfection, the cells were dissociated and labeled with biotinylated Rat Anti-HA antibody (Roche). The Anti-HA antibody was then detected with PE-conjugated streptavidin. The cells were analyzed on a BD FACSCalibur flow cytometer. The HA-tagged proteins were detected in the FL2 channel and GFP was detected in the FL1 channel.

FIG. 6 shows the results of Anti-HA Western blot analysis of 293-EBNA cells transfected with the HA-tagged FLJ32028 cDNAs. 293-EBNA cells were cotransfected with the HA-tagged FLJ32028 cDNAs in vector pCEP4 (Invitrogen) and the pEGFP plasmid (Clontech) which expresses the green fluorescent protein. After 48 hours of transfection, the cells were lysed in RIPA buffer. The lysates were run on 4-15% gradient polyacrylamide-SDS gels under non-reducing or reducing conditions and transferred to a nitrocellulose filter. The HA-tagged FLJ32028 proteins were detected by Western blot using an alkaline phosphatase-conjugated Rat Anti-HA antibody (Roche) and BCIP/NBT. Abbreviations used to label lanes are EV: lysate from cells transfected with empty vector, CT: lysate from cells transfected with the C-terminal HA-tagged FLJ32028 construct, NT: lysate from cells transfected with the N-terminal HA-tagged FLJ32028 construct.

FIG. 7 shows the nucleic acid sequence (SEQ. ID No. 2) encoding FLJ32028.

FIG. 8 shows the amino acid sequence (SEQ. ID No. 1) of FLJ32028.

FIG. 9 shows the results of titration of serum from mouse 5644 by ELISA and flow cytometry.

FIG. 10 shows the results of titration of serum from mouse 5640 by ELISA and flow cytometry.

FIG. 11 shows the results of flow cytometry establishing that antiserum raised against the FLJ30028 extracellular domain recognizes a cell surface antigen on primary CLL cells.

FIG. 12 shows the results of panning to isolate FLJ32028-specific antibodies from a mouse Fab phage display library. FIGS. 13 and 14 show the amino acid sequences of the FLJ32028-specific antibodies and the nucleic acid sequences encoding them.

FIGS. 15 and 16 show the variable region amino acid sequence alignments of FLJ32028-specific Fabs from the 5644 library.

FIG. 17 shows the binding of FLJ32028-specific Fab antibodies from the 5644 library to 293-EBNA cells transiently transfected with FLJ32028. Periplasmic fractions were prepared from E. coli cultures (strain TOP10F′) expressing the Fab antibodies. The periplasmic fractions were incubated with 293-EBNA cells expressing the full length FLJ32028 cDNA. After washing the cells, bound Fabs were detected with a PE-conjugated Goat Anti-Mouse IgG, F(ab′)₂ fragment-specific secondary antibody. The cells were analysed using a FACSCalibur flow cytometer.

FIGS. 18A-E show the flow cytometry results for some of the Fabs from the 5640 phage library that showed binding to FLJ32028 expressing cells.

FIG. 19 shows the deduced amino acid sequences of the heavy chains of the clones from the 5640 phage library that showed binding to FLJ32028 by flow cytometry.

### DETAILED DESCRIPTION OF THE PREFERRED EMBODIMENTS

I. Definitions

The terms “FLJ32028 polypeptide” and “FLJ32028” as used herein refers to specific polypeptide sequences as described herein encompass native sequence polypeptides and polypeptide variants (which are further defined herein). The FLJ32028 polypeptides described herein may be isolated from a variety of sources, such as from human tissue types or from another source, or prepared by recombinant or synthetic methods.

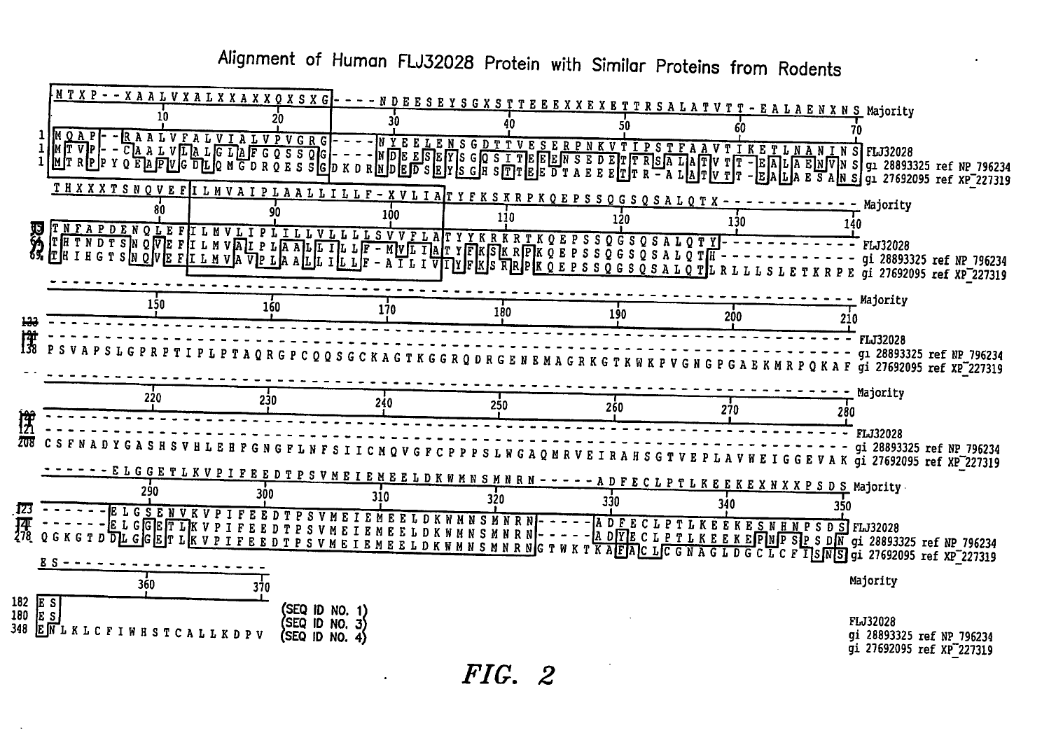

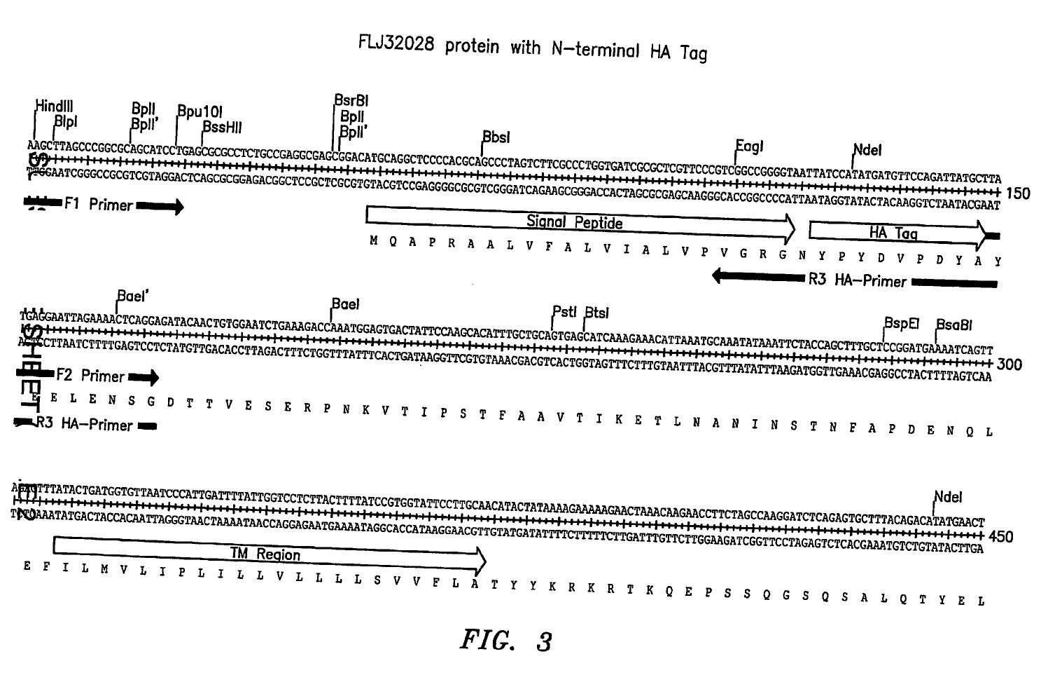

FLJ32028 is considered a hypothetical protein because it does not fall within any motif commonly known for proteins, i.e. it doesn't resemble any other known protein. An analysis of the known human FLJ32028 cDNA sequence shows that FLJ32028 is a Type Ia membrane protein. FLJ32028 has been identified as a potential CLL-associated marker by comparing FLJ32028 to public gene expression profile databases. These comparisons showed that FLJ32028 has no similarity to other human genes. However, a BLAST search of the Genbank database reveals a similarity with genes in both mouse and rat sequences. (See, FIG. 2.)

A “native sequence FLJ32028 polypeptide” comprises a polypeptide having the same amino acid sequence as the corresponding FLJ32028 polypeptide derived from nature. Such native sequence FLJ32028 polypeptides can be isolated from nature or can be produced by recombinant or synthetic means. The term “native sequence FLJ32028 polypeptide” specifically encompasses naturally-occurring truncated or secreted forms of the specific FLJ32028 polypeptide, naturally-occurring variant forms and naturally-occurring allelic variants of the polypeptide. In various embodiments, the native sequence FLJ32028 polypeptides disclosed herein are mature or full-length native sequence polypeptides comprising the full-length amino acids sequences shown in the accompanying figures.

The FLJ32028 polypeptide “extracellular domain” or “ECD” refers to a form of the FLJ32028 polypeptide which is essentially free of the transmembrane and cytoplasmic domains. Ordinarily, a FLJ32028 polypeptide ECD will have less than 1% of such transmembrane and/or cytoplasmic domains and preferably, will have less than 0.5% of such domains. It will be understood that any transmembrane domains identified for the FLJ32028 polypeptides are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a FLJ32028 polypeptide may contain from about 5 or fewer amino acids on either side of the transmembrane domain/extracellular domain boundary as identified herein and such polypeptides, with or without the associated signal peptide, and nucleic acid encoding them, are contemplated by the present invention.

The approximate location of the “signal peptides” of the various FLJ32028 polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“FLJ32028 polypeptide variant” means an active FLJ32028 polypeptide as defined above or below having at least about 80% amino acid sequence identity with a full-length native sequence FLJ32028 polypeptide sequence as disclosed herein, a FLJ32028 polypeptide sequence lacking the signal peptide as disclosed herein, an extracellular domain of a FLJ32028 polypeptide, with or without the signal peptide, as disclosed herein or any other fragment of a full-length FLJ32028 polypeptide sequence as disclosed herein. Such FLJ32028 polypeptide variants include, for instance, FLJ32028 polypeptides wherein one or more amino acid residues are added, or deleted, at the N- or C-terminus of the full-length native amino acid sequence. Ordinarily, a FLJ32028 polypeptide variant will have at least about 80% amino acid sequence identity, preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and most preferably at least about 99% amino acid sequence identity with a full-length native sequence FLJ32028 polypeptide sequence as disclosed herein, a FLJ32028 polypeptide sequence lacking the signal peptide as disclosed herein, an extracellular domain of a FLJ32028 polypeptide, with or without the signal peptide, as disclosed herein or any other specifically defined fragment of a full-length FLJ32028 polypeptide sequence as disclosed herein. Ordinarily, FLJ32028 variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 300 amino acids in length, or more.

“Percent (%) amino acid sequence identity” with respect to the FLJ32028 polypeptide sequences identified herein is defined as the percentage of amino acid residues in a candidate sequence that are identical with the amino acid residues in the specific FLJ32028 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

“FLJ32028 variant polynucleotide” or “FLJ32028 variant nucleic acid sequence” means a nucleic acid molecule which encodes an active FLJ32028 polypeptide as defined below and which has at least about 80% nucleic acid sequence identity with a nucleotide acid sequence encoding a full-length native sequence FLJ32028 polypeptide sequence as disclosed herein, a full-length native sequence FLJ32028 polypeptide sequence lacking the signal peptide as disclosed herein, an extracellular domain of a FLJ32028 polypeptide, with or without the signal peptide, as disclosed herein or any other fragment of a full-length FLJ32028 polypeptide sequence as disclosed herein. Ordinarily, a FLJ32028 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with a nucleic acid sequence encoding a full-length native sequence FLJ32028 polypeptide sequence as disclosed herein, a full-length native sequence FLJ32028 polypeptide sequence lacking the signal peptide as disclosed herein, an extracellular domain of a FLJ32028 polypeptide, with or without the signal sequence, as disclosed herein or any other fragment of a full-length FLJ32028 polypeptide sequence as disclosed herein. Variants do not encompass the native nucleotide sequence.

Ordinarily, FLJ32028 variant polynucleotides are at least about 30 nucleotides in length, often at least about 60 nucleotides in length, more often at least about 90 nucleotides in length, more often at least about 120 nucleotides in length, more often at least about 150nucleotides in length, more often at least about 180 nucleotides in length, more often at least about 210 nucleotides in length, more often at least about 240 nucleotides in length, more often at least about 270 nucleotides in length, more often at least about 300 nucleotides in length, more often at least about 450 nucleotides in length, more often at least about 600 nucleotides in length, more often at least about 900 nucleotides in length, or more.

“Percent (%) nucleic acid sequence identity” with respect to FLJ32028-encoding nucleic acid sequences identified herein is defined as the percentage of nucleotides in a candidate sequence that are identical with the nucleotides in the FLJ32028 nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software.

In other embodiments, FLJ32028 variant polynucleotides are nucleic acid molecules that encode an active FLJ32028 polypeptide and which are capable of hybridizing, preferably under stringent hybridization and wash conditions, to nucleotide sequences encoding a full-length FLJ32028 polypeptide as disclosed herein. FLJ32028 variant polypeptides may be those that are encoded by a FLJ32028 variant polynucleotide.

The term “positives”, in the context of sequence comparison performed as described above, includes residues in the sequences compared that are not identical but have similar properties (e.g. as a result of conservative substitutions, see Table 1 below). For purposes herein, the % value of positives is determined by dividing (a) the number of amino acid residues scoring a positive value between the FLJ32028 polypeptide amino acid sequence of interest having a sequence derived from the native FLJ32028 polypeptide sequence and the comparison amino acid sequence of interest (i.e., the amino acid sequence against which the FLJ32028 polypeptide sequence is being compared) as determined in the BLOSUM62 matrix of WU-BLAST-2 by (b) the total number of amino acid residues of the FLJ32028 polypeptide of interest.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the FLJ32028 polypeptide natural environment will not be present. Ordinarily, however, isolated polypeptide will be prepared by at least one purification step.

An “isolated” FLJ32028 polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-FLJ32028 monoclonal antibodies (including agonist, antagonist, and neutralizing antibodies), anti-FLJ32028 antibody compositions with polyepitopic specificity, single chain anti-FLJ32028 antibodies, and fragments of anti-FLJ32028 antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodiumcitrate), 50 mM sodiumphosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a FLJ32028 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a FLJ32028 polypeptide which retain a biological and/or an immunological activity of native or naturally-occurring FLJ32028, wherein “biological” activity refers to a biological function (either inhibitory or stimulatory) caused by a native or naturally-occurring FLJ32028 other than the ability to induce the production of an antibody against an antigenic epitope possessed by a native or naturally-occurring FLJ32028 and an “immunological” activity refers to the ability to induce the production of an antibody against an antigenic epitope possessed by a native or naturally-occurring FLJ32028.

The term “antagonist” is used in the broadest sense, and includes any molecule that partially or fully blocks, inhibits, or neutralizes a biological activity of a native FLJ32028 polypeptide disclosed herein. In a similar manner, the term “agonist” is used in the broadest sense and includes any molecule that mimics a biological activity of a native FLJ32028 polypeptide disclosed herein. Suitable agonist or antagonist molecules specifically include agonist or antagonist antibodies or antibody fragments, fragments or amino acid sequence variants of native FLJ32028 polypeptides, peptides, antisense oligonucleotides, small organic molecules, etc. Methods for identifying agonists or antagonists of a FLJ32028 polypeptide may comprise contacting a FLJ32028 polypeptide with a candidate agonist or antagonist molecule and measuring a detectable change in one or more biological activities normally associated with the FLJ32028 polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments.

Papain digestion of antibodies FLJ32028duces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., FLJ32028c. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a FLJ32028 polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

II. Compositions and Methods of the Invention

The present disclosure provides newly identified and isolated nucleotide sequences encoding polypeptides referred to in the present application as FLJ32028 polypeptides. In particular, cDNAs encoding various FLJ32028 polypeptides have been identified and isolated, as disclosed in further detail in the Examples below.

As disclosed in the Examples below, various cDNA clones have been prepared. The actual nucleotide sequences of those clones can readily be determined by the skilled artisan by sequencing of the deposited clone using routine methods in the art. The predicted amino acid sequence can be determined from the nucleotide sequence using routine skill. For the FLJ32028 polypeptides and encoding nucleic acids described herein, What is believed to be the reading frame best identifiable with the sequence information available at the time has been identified.

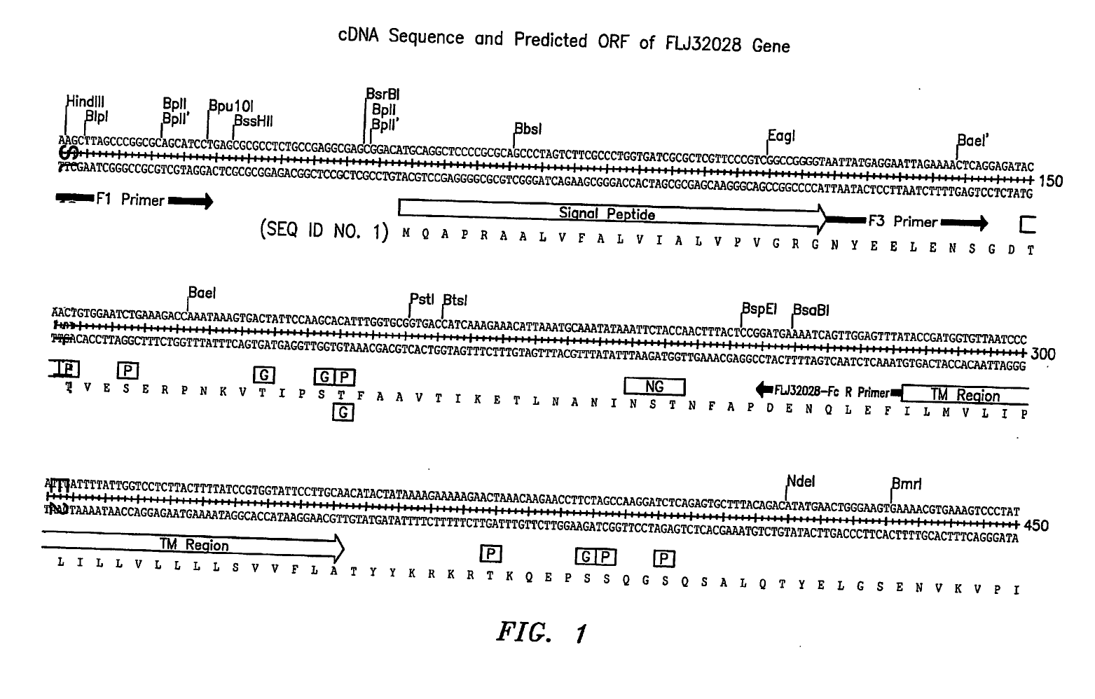

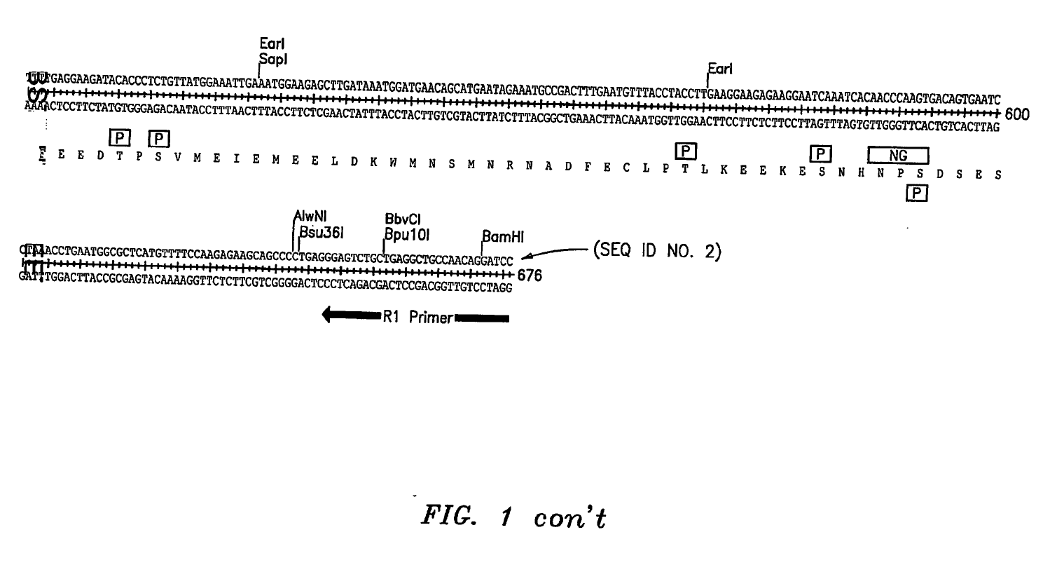

The full-length native sequence FLJ32028 is shown in FIG. 1 and SEQ ID NO: 1. In addition to the full-length native sequence FLJ32028 polypeptides described herein, it is contemplated that FLJ32028 variants can be prepared. FLJ32028 variants can be prepared by introducing appropriate nucleotide changes into the FLJ32028 DNA, and/or by synthesis of the desired FLJ32028 polypeptide. Those skilled in the art will appreciate that amino acid changes may alter post-translational processes of the FLJ32028, such as changing the number or position of glycosylation sites or altering the membrane anchoring characteristics.

Variations in the native full-length sequence FLJ32028 or in various domains of the FLJ32028 described herein, can be made, for example, using any of the techniques and guidelines for conservative and non-conservative mutations set forth, for instance, in U.S. Pat. No. 5,364,934. Variations may be a substitution, deletion or insertion of one or more codons encoding the FLJ32028 that results in a change in the amino acid sequence of the FLJ32028 as compared with the native sequence FLJ32028. Optionally the variation is by substitution of at least one amino acid with any other amino acid in one or more of the domains of the FLJ32028. Guidance in determining which amino acid residue may be inserted, substituted or deleted without adversely affecting the desired activity may be found by comparing the sequence of the FLJ32028 with that of homologous known Protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

FLJ32028 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the FLJ32028 polypeptide.

FLJ32028 fragments may be prepared by any of a number of conventional techniques. Desired peptide fragments may be chemically synthesized. An alternative approach involves generating FLJ32028 fragments by enzymatic digestion, e.g., by treating the protein with an enzyme known to cleave Proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, FLJ32028 polypeptide fragments share at least one biological and/or immunological activity with the native FLJ32028 polypeptide disclosed herein.

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; | |
| | phe; | |
| | norleucine | leu |
| Leu (L) | norleucine; ile; val; | |
| | met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | |
| | ala; norleucine | leu |

Substantial modifications in function or immunological identity of the FLJ32028 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain Properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the FLJ32028 variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244: 1081-1085 (1989)). Alanine is also typically preferred because it is the most common amino acid. Further, it is frequently found in both buried and exposed positions (Creighton, The Proteins, (W. H. Freeman & Co., N.Y.); Chothia, J. Mol. Biol., 150:1 (1976)). If alanine substitution does not yield adequate amounts of variant, an isoteric amino acid can be used.

Covalent modifications of FLJ32028 are included within the scope of this disclosure. One type of covalent modification includes reacting targeted amino acid residues of a FLJ32028 polypeptide with an organic derivatizing agent that is capable of reacting with selected side chains or the N- or C-terminal residues of the FLJ32028. Derivatization with bifunctional agents is useful, for instance, for crosslinking FLJ32028 to a water-insoluble support matrix or surface for use in the method for purifying anti-FLJ32028 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification of the FLJ32028 polypeptide included within the scope of this invention comprises altering the native glycosylation pattern of the polypeptide. “Altering the native glycosylation pattern” is intended for purposes herein to mean deleting one or more carbohydrate moieties found in native sequence FLJ32028 (either by removing the underlying glycosylation site or by deleting the glycosylation by chemical and/or enzymatic means), and/or adding one or more glycosylation sites that are not present in the native sequence FLJ32028. In addition, the phrase includes qualitative changes in the glycosylation of the native Proteins, involving a change in the nature and proportions of the various carbohydrate moieties present.

Addition of glycosylation sites to the FLJ32028 polypeptide may be accomplished by altering the amino acid sequence. The alteration may be made, for example, by the addition of, or substitution by, one or more serine or threonine residues to the native sequence FLJ32028 (for O-linked glycosylation sites). The FLJ32028 amino acid sequence may optionally be altered through changes at the DNA level, particularly by mutating the DNA encoding the FLJ32028-polypeptide at preselected bases such that codons are generated that will translate into the desired amino acids.

Another means of increasing the number of carbohydrate moieties on the FLJ32028 polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the FLJ32028 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of FLJ32028 comprises linking the FLJ32028 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The FLJ32028 may also be modified in a way to form a chimeric molecule comprising FLJ32028 fused to another, heterologous polypeptide or amino acid sequence.

In one embodiment, such a chimeric molecule comprises a fusion of the FLJ32028 with a tag polypeptide which provides an epitope to which an anti-tag antibody can selectively bind. The epitope tag is generally placed at the amino- or carboxyl-terminus of the FLJ32028. The presence of such epitope-tagged forms of the FLJ32028 can be detected using an antibody against the tag polypeptide. Also, provision of the epitope tag enables the FLJ32028 to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985); and the Herpes Simplex virus glycoProtein D (gD) tag and its antibody (Paborsky et al., Protein Engineering, 2(6):547-553 (1990)). Other tag polypeptides include the Flag-peptide (Hopp et al., BioTechnology, 6:1204-1210 (1988)); the KT3 epitope peptide (Martin et al., Science, 255:192-194 (1992)); an α-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

In an alternative embodiment, the chimeric molecule may comprise a fusion of the FLJ32028 with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a FLJ32028 polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

The production of FLJ32028 can be achieved by culturing cells transformed or transfected with a vector containing FLJ32028 nucleic acid. It is, of course, contemplated that alternative methods, which are well known in the art, may be employed to prepare FLJ32028. For instance, the FLJ32028 sequence, or portions thereof, may be produced by direct peptide synthesis using solid-phase techniques (see, e.g., Stewart et al., Solid-Phase Peptide Synthesis, W. H. Freeman Co., San Francisco, Calif. (1969); Merrifield, J. Am. Chem. Soc., 85:2149-2154 (1963)). In vitro Protein synthesis may be performed using manual techniques or by automation. Automated synthesis may be accomplished, for instance, using an Applied Biosystems Peptide Synthesizer (Foster City, Calif.) using manufacturer's instructions. Various portions of the FLJ32028 may be chemically synthesized separately and combined using chemical or enzymatic methods to produce the full-length FLJ32028.

DNA encoding FLJ32028 may be obtained from a cDNA library prepared from tissue believed to possess the FLJ32028 mRNA and to express it at a detectable level. Accordingly, human FLJ32028 DNA can be conveniently obtained from a cDNA library prepared from human tissue, such as described in the Examples. The FLJ32028-encoding gene may also be obtained from a genomic library or by known synthetic procedures (e.g., automated nucleic acid synthesis).

Libraries can be screened with probes (such as antibodies to the FLJ32028 or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding FLJ32028 is to use PCR methodology (Sambrook et al., supra; Dieffenbach et al., PCR Primer: A Laboratory Manual (Cold Spring Harbor Laboratory Press, 1995)).

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

Host cells are transfected or transformed with expression or cloning vectors described herein for FLJ32028 production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published Jun. 29, 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, FLJ32028teus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. These examples are illustrative rather than limiting. Strain W3 110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3phoA E15 (argF-lac) 169 degP ompTkan^{r}; E. coli W3110 strain 37D6, which has the complete genotype tonA ptr3 phoA E15 (argF-lac) 169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990. Alternatively, in vitro methods of cloning, e.g., PCR or other nucleic acid polymerase reactions, are suitable.

In addition to prokaryotes,, eukaryotic microbes such as filamentous fungi or yeast are suitable cloning or expression hosts for FLJ32028-encoding vectors. Saccharomyces cerevisiae is a commonly used lower eukaryotic host microorganism. Others include Schizosaccharomyces pombe (Beach and Nurse, Nature, 290: 140 (1981); EP 139,383 published May 2, 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis EP 394,538 published Oct. 32, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81: 1470-1474 (1984)) and A. niger (Kelly and Hynes, EMBO J., 4:475479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated FLJ32028 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

The nucleic acid (e.g., cDNA or genomic DNA) encoding FLJ32028 may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The FLJ32028 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the FLJ32028-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences may be used to direct secretion of the protein, such as signal sequences from secreted polypeptides of the same or related species, as well as viral secretory leaders. In an alternative embodiment white insect cell expression is used, the signal sequence can be from the baculovirus envelope protein gp67.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode Proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the FLJ32028-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene FLJ32028vides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the FLJ32028-encoding nucleic acid sequence to direct mRNA synthesis. Promoters recognized by a variety of potential host cells are well known. Promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also will contain a Shine-Dalgarno (S.D.) sequence operably linked to the DNA encoding FLJ32028.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

FLJ32028 transcription from vectors in mammalian host cells is controlled, for example, by Promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin Promoter, and from heat-shock promoters, provided such promoters are compatible with the host cell systems.

Transcription of a DNA encoding the FLJ32028 by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the FLJ32028 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding FLJ32028.

Still other methods, vectors, and host cells suitable for adaptation to the synthesis of FLJ32028 in recombinant vertebrate cell culture are described in Gething et al., Nature, 293:620-625 (1981); Mantei et al., Nature, 281:4046 (1979); EP 117,060; and EP 117,058.

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. In a particularly useful embodiment, real time PCR is used to quantitatively measure expression wherein fluorescently labeled primers are used to conduct PCR and fluorescence is measured over time. (See, e.g., Gibson et al., Genome Research 6 (10), pages 995-1001 (1996); and Heid et al. Genome Research 6 (10), pages 986-994 (1996). Alternatively, antibodies may be employed that can recognize specific duplexes, including DNA duplexes, RNA duplexes, and DNA-RNA hybrid duplexes or DNA-Protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence FLJ32028 polypeptide or against a synthetic peptide based on the DNA sequences provided herein or against exogenous sequence fused to FLJ32028 DNA and encoding a specific antibody epitope.

Forms of FLJ32028 may be recovered from culture medium or from host cell lysates. If membrane-bound, it can be released from the membrane using a suitable detergent solution (e.g. Triton-X 100) or by enzymatic cleavage. Cells employed in expression of FLJ32028 can be disrupted by various physical or chemical means, such as freeze-thaw cycling, sonication, mechanical disruption, or cell lysing agents.

It may be desired to purify FLJ32028 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; Protein A Sepharose columns to remove contaminants such as IgG; and metal chelating columns to bind epitope-tagged forms of the FLJ32028. Various methods of Protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular FLJ32028 produced.

Nucleotide sequences (or their complement) encoding FLJ32028 have various applications in the art of molecular biology, including uses as hybridization probes, in chromosome and gene mapping and in the generation of anti-sense RNA and DNA. FLJ32028 nucleic acid will also be useful for the preparation of FLJ32028 polypeptides by the recombinant techniques described herein.

The full-length native sequence FLJ32028 gene, or portions thereof, may be used as hybridization probes for a cDNA library to isolate the full-length FLJ32028 cDNA or to isolate still other cDNAs (for instance, those encoding naturally-occurring variants of FLJ32028 or FLJ32028 from other species) which have a desired sequence identity to the native FLJ32028 sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including Promoters, enhancer elements and introns of native sequence FLJ32028. By way of example, a screening method will comprise isolating the coding region of the FLJ32028 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the FLJ32028 gene of the present invention can be used to screen libraries of human cDNA, genomic DNA or mRNA to determine which members of such libraries the probe hybridizes to.

Any EST sequences disclosed in the present application may similarly be employed as probes, using the methods disclosed herein.

Other useful fragments of the FLJ32028 nucleic acids include antisense or sense oligonucleotides comprising a singe-stranded nucleic acid sequence (either RNA or DNA) capable of binding to target FLJ32028 mRNA (sense) or FLJ32028 DNA (antisense) sequences. Antisense or sense oligonucleotides, according to the present invention, comprise a fragment of the coding region of FLJ32028 DNA. Such a fragment generally comprises at least about 14 nucleotides, preferably from about 14 to 30 nucleotides. The ability to derive an antisense or a sense oligonucleotide, based upon a cDNA sequence encoding a given Protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of FLJ32028 proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Alternatively, a fluorescent microsphere (“covasphere”) ligand:receptor binding assay can be used such as those disclosed in Brown et al., “Eur. J. Immunol. 25 (12), pages 3222-3228 (1995) or Preston et al., Eur. J. Immunol. 27 (8), pages 1911-1918 (1997). Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

The probes may also be employed in PCR techniques to generate a pool of sequences for identification of closely related FLJ32028 coding sequences.

Nucleotide sequences encoding a FLJ32028 can also be used to construct hybridization probes for mapping the gene which encodes that FLJ32028 and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for FLJ32028 encode a protein which binds to another protein (example, where the FLJ32028 functions as a receptor), the FLJ32028 can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor FLJ32028 can be used to isolate correlative ligand(s). Screening assays can be designed to find lead compounds that mimic the biological activity of a native FLJ32028 or a receptor for FLJ32028. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including Protein-Protein binding assays, biochemical screening assays, immunoassays and cell based assays, which are well characterized in the art.

Nucleic acids which encode FLJ32028 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding FLJ32028 can be used to clone genomic DNA encoding FLJ32028 in accordance with established techniques and the genomic sequences used to generate transgenic animals that contain cells which express DNA encoding FLJ32028. Methods for generating transgenic animals, particularly animals such as mice or rats, have become conventional in the art and are described, for example, in U.S. Pat. Nos. 4,736,866 and 4,870,009. Typically, particular cells would be targeted for FLJ32028 transgene incorporation with tissue-specific enhancers. Transgenic animals that include a copy of a transgene encoding FLJ32028 introduced into the germ line of the animal at an embryonic stage can be used to examine the effect of increased expression of DNA encoding FLJ32028. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of FLJ32028 can be used to construct a FLJ32028 “knock out” animal which has a defective or altered gene encoding FLJ32028 as a result of homologous recombination between the endogenous gene encoding FLJ32028 and altered genomic DNA encoding FLJ32028 introduced into an embryonic stem cell of the animal. For example, cDNA encoding FLJ32028 can be used to clone genomic DNA encoding FLJ32028 in accordance with established techniques. A portion of the genomic DNA encoding FLJ32028 can be deleted or replaced with another gene, such as a gene encoding a selectable marker which can be used to monitor integration. Typically, several kilobases of unaltered flanking DNA (both at the 5′ and 3′ ends) are included in the vector (see e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected (see e.g., Li et al., Cell, 69:915 (1992)). The selected cells are then injected into a blastocyst of an animal (e.g., a mouse or rat) to form aggregation chimeras (see e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the FLJ32028 polypeptide.

Nucleic acid encoding the FLJ32028 polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves-the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 (1986)). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat Protein-liposome mediated transfection (Dzau et al., Trends in Biotechnology 11, 205-210 (1993)). In some situations it is desirable to provide the nucleic acid source with an agent that targets the target cells, such as an antibody specific for a cell surface membrane Protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The FLJ32028 polypeptides described herein may also be employed as molecular weight markers for protein electrophoresis purposes and the isolated nucleic acid sequences may be used for recombinantly expressing those markers.

The nucleic acid molecules encoding the FLJ32028 polypeptides or fragments thereof described herein are useful for chromosome identification. In this regard, there exists an ongoing need to identify new chromosome markers, since relatively few chromosome marking reagents, based upon actual sequence data are presently available. Each FLJ32028 nucleic acid molecule of the present invention can be used as a chromosome marker.

The FLJ32028 polypeptides and nucleic acid molecules of the present invention may also be used for tissue typing; wherein the FLJ32028 polypeptides of the present invention may be differentially expressed in one tissue as compared to another. FLJ32028 nucleic acid molecules will find use for generating probes for PCR, Northern analysis, Southern analysis and Western analysis.

The FLJ32028 polypeptides described herein may also be employed as therapeutic agents. The FLJ32028 polypeptides of the present invention can be formulated according to known methods to prepare pharmaceutically useful compositions, whereby the FLJ32028 product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a FLJ32028 polypeptide or agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a FLJ32028 polypeptide is desired in a formulation with release characteristics suitable for the treatment of any disease or disorder requiring administration of the FLJ32028 polypeptide, microencapsulation of the FLJ32028 polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439462; WO 97/03692, WO 96/40072, WO 96/07399; and U.S. Pat. No. 5,654,010.

The sustained-release formulations of these proteins were developed using polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

This disclosure encompasses methods of screening compounds to identify those that mimic the FLJ32028 polypeptide (agonists) or prevent the effect of the FLJ32028 polypeptide (antagonists). Screening assays for antagonist drug candidates are designed to identify compounds that bind or complex with the FLJ32028 polypeptides encoded by the genes identified herein, or otherwise interfere with the interaction of the encoded polypeptides with other cellular Proteins. Such screening assays will include assays amenable to high-throughput screening of chemical libraries, making them particularly suitable for identifying small molecule drug candidates.

The assays can be performed in a variety of formats, including Protein-Protein binding assays, biochemical screening assays, immunoassays, and cell-based assays, which are well characterized in the art.

All assays for antagonists are common in that they call for contacting the drug candidate with a FLJ32028 polypeptide encoded by a nucleic acid identified herein under conditions and for a time sufficient to allow these two components to interact.

In binding assays, the interaction is binding and the complex formed can be isolated or detected in the reaction mixture. In a particular embodiment, the FLJ32028 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the FLJ32028 polypeptide and drying. Alternatively, an immobilized antibody, e.g., a monoclonal antibody, specific for the FLJ32028 polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular FLJ32028 polypeptide encoded by a gene identified herein, its interaction with that polypeptide can be assayed by methods well known for detecting Protein-Protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London,), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via Protein-Protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a FLJ32028 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the FLJ32028 polypeptide may be added to a cell along with the compound to be screened for a particular activity and the ability of the compound to inhibit the activity of interest in the presence of the FLJ32028 polypeptide indicates that the compound is an antagonist to the FLJ32028 polypeptide. Alternatively, antagonists may be detected by combining the FLJ32028 polypeptide and a potential antagonist with membrane-bound FLJ32028 polypeptide receptors or recombinant receptors under appropriate conditions for a competitive inhibition assay. The FLJ32028 polypeptide can be labeled, such as by radioactivity, such that the number of FLJ32028 polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the FLJ32028 polypeptide and a cDNA library created from this RNA is divided into pools and used to transfect COS cells or other cells that are not responsive to the FLJ32028 polypeptide. Transfected cells that are grown on glass slides are exposed to labeled FLJ32028 polypeptide. The FLJ32028 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled FLJ32028 polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled FLJ32028 polypeptide in the presence of the candidate compound. The ability of the compound to enhance or block this interaction could then be measured.

More specific examples of potential antagonists include an oligonucleotide that binds to the fusions of immunoglobulin with FLJ32028 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related Protein, for example, a mutated form of the FLJ32028 polypeptide that recognizes the receptor but imparts no effect, thereby competitively inhibiting the action of the FLJ32028 polypeptide.

Another potential FLJ32028 polypeptide antagonist is an antisense RNA or DNA construct prepared using antisense technology, where, e.g., an antisense RNA or DNA molecule acts to block directly the translation of mRNA by hybridizing to targeted mRNA and preventing Protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature FLJ32028 polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the Production of the FLJ32028 polypeptide. The antisense RNA oligonucleotide hybridizes to the mRNA in vivo and blocks translation of the mRNA molecule into the FLJ32028 polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the FLJ32028 polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the FLJ32028 polypeptide, thereby blocking the normal biological activity of the FLJ32028 polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Uses of the herein disclosed molecules may also be based upon positive functional assay hits.

### Anti-FLJ32028 Antibodies

The present disclosure further provides anti-FLJ32028 antibodies. Exemplary antibodies include polyclonal, monoclonal, humanized, bispecific, and heteroconjugate antibodies.

The anti-FLJ32028 antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the FLJ32028 polypeptide or a fusion protein thereof. It may be useful to conjugate the immunizing agent to a protein known to be immunogenic in the mammal being immunized. In another embodiment, the mammal can be immunized with cells (such as, for example, 293 EBNA cells) which are transfected with a vector expressing the protein. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The anti-FLJ32028 antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the FLJ32028 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guainine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against FLJ32028. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification Procedures such as, for example, Protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The anti-FLJ32028 antibodies in accordance with this disclosure may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Bispecific antibodies are monoclonal, preferably human or humanized, antibodies that have binding specificities for at least two different antigens. In the present case, one of the binding specificities is for the FLJ32028, the other one is for any other antigen, and preferably for a cell-surface Protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) Produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 3, 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exy. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been Produced using leucine zippers. Kostelny et al., J. Immunol. 148(5):1547-1553 (1992). The leucine zipper peptides from the Fos and Jun Proteins were linked to the Fab′ portions of two different antibodies by gene fusion. The antibody homodimers were reduced at the hinge region to form monomers and then re-oxidized to form the antibody heterodimers. This method can also be utilized for the Production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given FLJ32028 polypeptide herein. Alternatively, an anti-FLJ32028 polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular FLJ32028 polypeptide. Bispecific antibodies may also be used to localize cytotoxic agents to cells which express a particular FLJ32028 polypeptide. These antibodies possess a FLJ32028-binding arm and an arm which binds a cytotoxic agent or a radionuclide chelator, such as EOTUBE, DPTA, DOTA, or TETA. Another bispecific antibody of interest binds the FLJ32028 polypeptide and further binds tissue factor (TF).

Heteroconjugate antibodies are also within the scope of the present disclosure. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies may be prepared in vitro using known methods in synthetic Protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design, 3: 219-230 (1989).

The disclosure also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii Proteins, dianthin Proteins, Phytolaca americana Proteins (PAPI, PAPII, and PAP-S), momordica charantia inhibitor, curcin, crotin, saponaria officinalis inhibitor, gelonin, mitogellin, restrictocin, phenomycin, enomycin, and the tricothecenes. A variety of radionuclides are available for the Production of radioconjugated antibodies. Examples include ²¹²Bi, ¹³¹I, ¹³¹In, 90Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosure can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Antibodies specifically binding a FLJ32028 polypeptide identified herein, as well as other molecules identified by the screening assays disclosed hereinbefore, can be administered for the treatment of various disorders in the form of pharmaceutical compositions.

If the FLJ32028 polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target Protein sequence. Such peptides can be synthesized chemically and/or produced by recombinant DNA technology. See, e.g., Marasco et al., Proc. Natl. Acad. Sci. USA, 90: 7889-7893 (1993). The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release Proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiodisulfide interchange, stabilization may be achieved by modifying sulfhydryl residues, lyophilizing from acidic solutions, controlling moisture content, using appropriate additives, and developing specific polymer matrix compositions.

The anti-FLJ32028 antibodies of the invention have various utilities. For example, anti-FLJ32028 antibodies may be used in diagnostic assays for FLJ32028, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-FLJ32028 antibodies also are useful for the affinity purification of FLJ32028 from recombinant cell culture or natural sources. In this process, the antibodies against FLJ32028 are immobilized on a suitable support, such a Sephadex resin or filter paper, using methods well known in the art. The immobilized antibody then is contacted with a sample containing the FLJ32028 to be purified, and thereafter the support is washed with a suitable solvent that will remove substantially all the material in the sample except the FLJ32028, which is bound to the immobilized antibody. Finally, the support is washed with another suitable solvent that will release the FLJ32028 from the antibody.

The following examples are offered for illustrative purposes only, and are not intended to limit the scope of the present invention in any way.

All patent and literature references cited in the present specification are hereby incorporated by reference in their entirety.

Commercially available reagents referred to in the following examples were used according to manufacturer's instructions unless otherwise indicated. The source of cells identified in the following examples, and throughout the specification, by ATCC accession numbers is the American Type Culture Collection, Manassas, Va.

### EXAMPLES

FLJ32028 cDNA was cloned from mRNA isolated from primary CLL cells using conventional reverse transcription PCR. To demonstrate that the cDNA encoded a Type Ia membrane protein, epitope tags were fused to either the predicted N-terminal or C-terminal end of the ORF. Western blot showed that both proteins were expressed, but flow cytometry reveals that only the N-terminal tag was accessible to the anti-tag antibody in intact cells. These results show that the N-terminal end of the protein is extracellular and the C-terminal end is cytoplasmic.

To generate monoclonal antibodies to the extracellular domain of FLJ32028, the extracellular domain is produced in the baculovirus system as an Fc fusion protein. This FLJ32028-Fc fusion protein and 293-EBNA cells expressing full-length FLJ32028 is used to immunize mice. Spleens from the immunized mice are used to produce Fab antibody phage libraries. Monoclonal Fab antibodies to the FLJ32028 extracellular domain are selected from the libraries by panning on the FLJ32028-Fc fusion protein.

The Fab antibodies are used to analyze the expression of FLJ32028 on the surface of primary CLL cells as well as on normal human cells and tissues. Antibodies that show some specificity for CLL cells are converted to chimeric mouse-human full IgG antibodies and assayed for their ability to kill CLL cells in vitro and in vivo.

### Example 1

### FLJ32028 Identification

To identify mRNAs specific for, or associated with, B-CLL, the lymphochip cDNA microarray data from the Leukemia/Lymphoma Molecular Profiling Project (LLMPP, http://llmpp.nih.gov) was browsed, using the GeneExplorer web application. mRNAs that were expressed at higher levels in CLL cells relative to normal lymphocytes were analyzed for the presence of ORFs encoding potential transmembrane proteins. ORFs were analyzed using the post-translational modification and topology prediction tools on the ExPASy Molecular Biology Server (http://us.expasy.org). See, Alizadeh et al. Distinct types of diffuse large B-cell lymphomas identified by gene expression profiling. Nature 403, 503-511 (2000). Web supplement http://llmpp.nih.gov/lymphoma; Rosenwald et al. Relation of gene expression phenotype to immunoglobulin mutation genotype in B cell chronic lymphocytic leukemia. J. Exp. Med. 194, 1639-1647 (2001). Web supplement http://llmpp.nih.gov/cll.

One CLL-associated cDNA that was identified was the gene for the hypothetical protein FLJ32028 (Genbank accession number XM_{—}114380.2). This gene is located on human chromosome band 4q31.3 and contains an ORF for a 183 amino acid protein. Analysis of the polypeptide sequence with the PSORT (http://psort.nibb.ac jp/), SIGNALP (http://www.cbs.dtu.dk/services/SignalP/), and TMHMM (http://www.cbs.dtu.dk/services/TMHMM/) topology prediction tools shows the presence of a cleavable N-terminal signal sequence and a single transmembrane region (FIG. 1). Therefore, FLJ32028 is a Type Ia membrane protein. Analysis of the polypeptide sequence with the PROSITE http://us.expasy.org/prosite/), NetNGlyc (http://www.cbs.dtu.dk/services/NetNGlyc/), NetOGlyc (http://www.cbs.dtu.dk/services/NetOGlyc/), and NetPhos (http://www.cbs.dtu.dk/services/NetPhos/) postranslational modification tools predicted two potential N-linked glycosylation sites, four potential O-linked glycosylation sites, and 12 potential serine/threonine phosphorylation sites (FIG. 1). No potential tyrosine phosphorylation sites were found.

A BLAST search of the Genbank DNA and protein sequence databases was performed to identify sequences with similarity to the hypothetical FLJ32028 protein (http://www.ncbi.nlm.nih.gov/BLAST/). No similar sequences were found in the human genome but two cDNAs with significant similarity to FLJ32028 were found in mouse and rat (FIG. 2). Interestingly, the C-terminal portion of human FLJ32028 of the predicted transmembrane region was highly conserved in the hypothetical rodent proteins, whereas the N-terminal portion of human FLJ32028 including the predicted signal peptide and transmembrane region was poorly conserved (FIG. 2).

### Example 2

### Protein Isolation

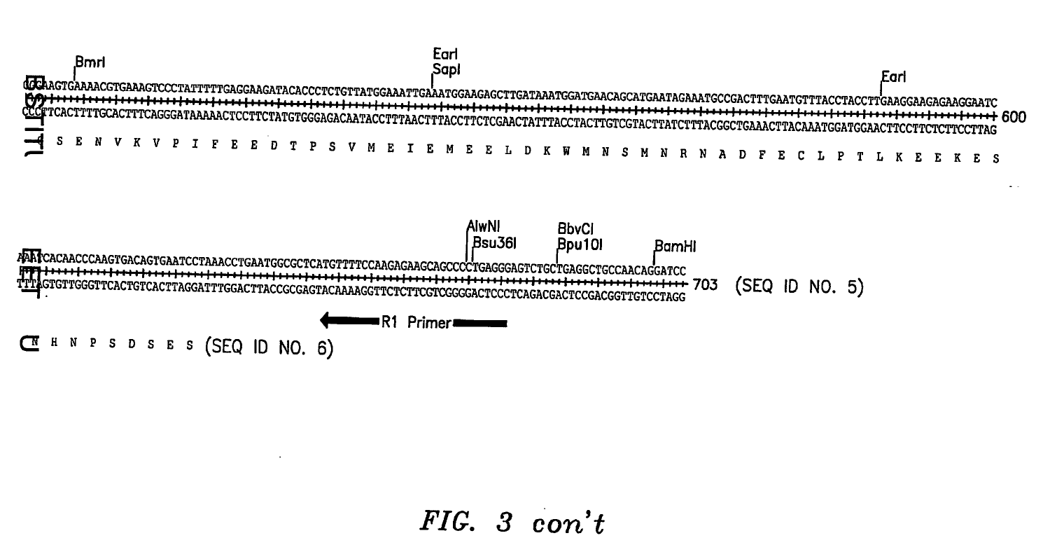

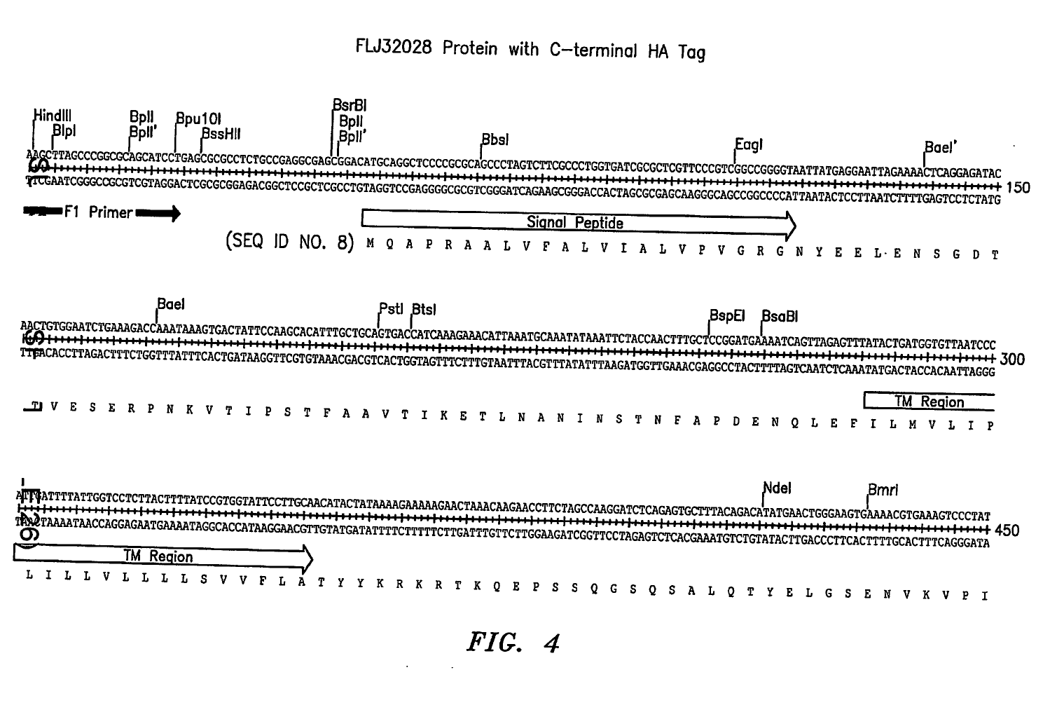

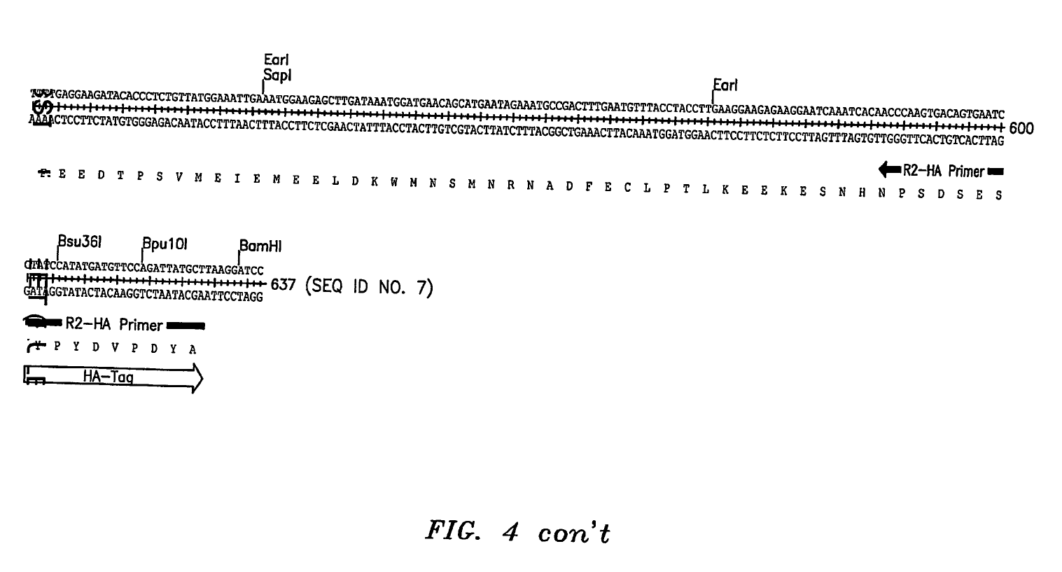

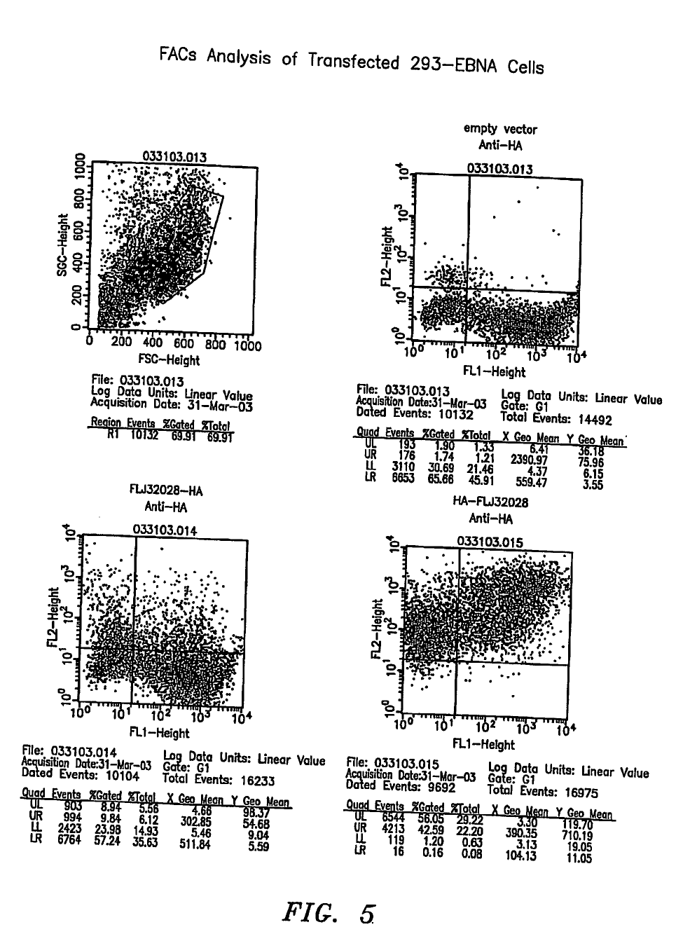

An FLJ32028 cDNA was isolated from primary CLL cells by RT-PCR and cloned into the mammalian expression vector pCEP4 (Invitrogen Corporation, Carlsbad, Calif.). To demonstrate that the FLJ32028 protein is a Type Ia membrane protein, the N-terminal end is shown to be extracellular and the C-terminal end to be cytoplasmic. To determine the membrane topology of FLJ32028, hemagglutinin protein (“HA”) epitope tags were inserted into the cDNA at either the N-terminal or C-terminal ends of the ORF (FIGS. 3 and 4) (Roche Diagnostics Corporation, Roche Applied Science, Indianapolis, Ind.).

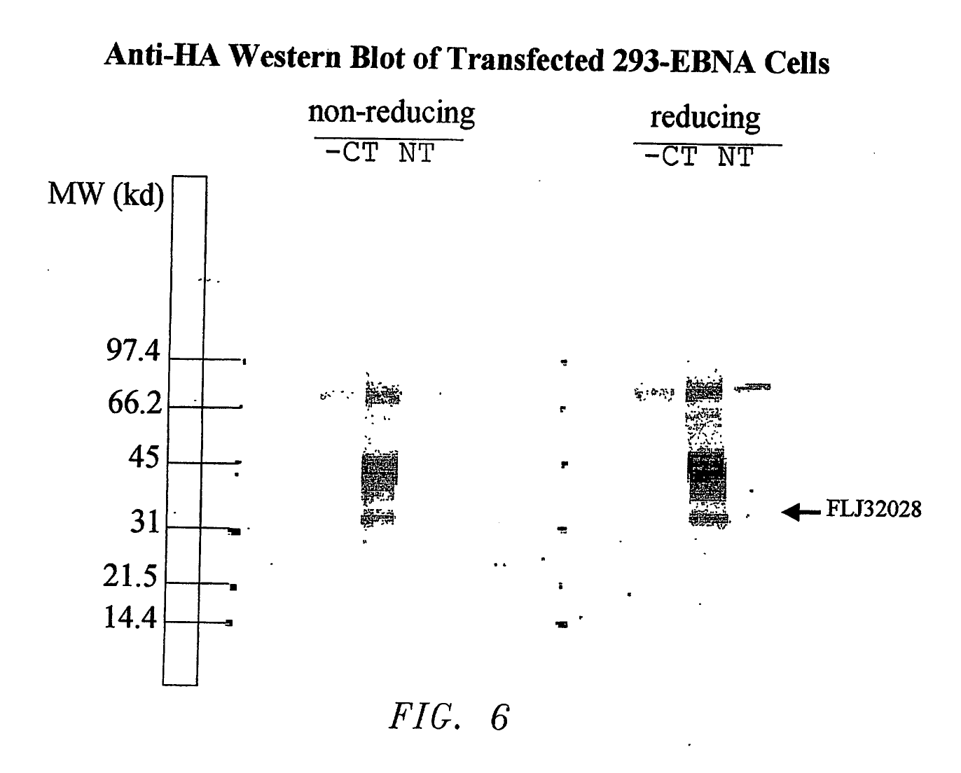

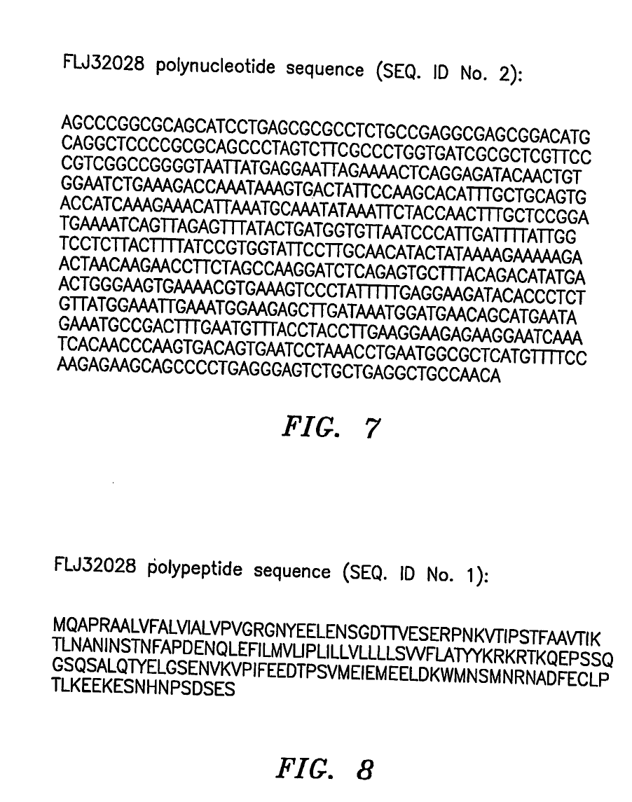

The two epitope-tagged FLJ32028 proteins were transiently expressed in 293-EBNA cells and analyzed by flow cytometry and Western blot using a biotinylated anti-HA tag antibody (FIGS. 5 and 6). 293-EBNA cells (from Invitrogen Corp., Carlsbad, Calif.) were cotransfected with the HA-tagged FLJ32028 cDNAs in vector pCEP4 (Invitrogen Corporation, Carlsbad, Calif.) and the pEGFP plasmid (BD Biosciences Clontech, Palo Alto, Calif.) which expresses the GFP. After 48 hours of transfection, the cells were lysed in RIPA buffer. The lysates were run on 4-15% gradient polyacrylamide-SDS gels under non-reducing or reducing conditions and transferred to a nitrocellulose filter. The HA-tagged FLJ32028 proteins were detected by Western blot using an alkaline phosphatase-conjugated Rat Anti-HA antibody (Roche) and BCIP/NBT substrate kit (BD Biosciences Pharmingen, San Diego, Calif.). (FIG. 6) Abbreviations used to label lanes are EV: lysate from cells transfected with empty vector, CT: lysate from cells transfected with the C-terminal HA-tagged FLJ32028 construct, NT: lysate from cells transfected with the N-terminal HA-tagged FLJ32028 construct.

Flow cytometric analysis of 293-EBNA cells transiently transfected with the HA epitope-tagged FLJ32028 cDNAs. 293-EBNA cells were co-transfected with the HA-tagged FLJ32028 cDNAs in vector pCEP4 (Invitrogen Corporation, Carlsbad, Calif.) and the pEGFP plasmid (BD Biosciences Clontech, Palo Alto, Calif.) which expresses the GFP. After 48 hours of transfection, the cells were dissociated and labeled with biotinylated Rat Anti-HA antibody (Roche). The Anti-HA antibody was then detected with PE-conjugated streptavidin. The cells were analyzed on a BD FACSCalibur flow cytometer. The HA-tagged proteins were detected in the FL2 channel and GFP was detected in the FL1 channel.

The green fluorescent protein (“GFP”) was co-expressed as a marker to distinguish transfected from nontransfected cells. 85% of the transfected GFP+ cells were negative for HA when intact cells expressing the C-terminal tagged FLJ32028 were labeled with the biotinylated anti-HA antibody and streptavidin-PE (Becton Dickinson “BD”, 1 Becton Drive, Franklin Lakes, N.J. USA 07417) (FIG. 5). This shows that the C-terminal HA tag is not accessible to the antibody in intact cells and the cytoplasmic location of the C-terminal end. In contrast, when intact cells expressing the N-terminal tagged FLJ32028 were labeled with the biotinylated anti-HA antibody and streptavidin-PE, greater than 99% of the transfected GFP+ cells were strongly positive for HA (FIG. 5). This shows that the N-terminal HA tag is completely accessible to the antibody in intact cells and the extracellular location of the N-terminal end. To verify that both tagged proteins were being expressed, lysates were prepared from the transfected cells and analyzed by Western blot (FIG. 6). Both proteins were detected using the biotinylated anti-HA antibody and alkaline phosphatase-conjugated streptavidin. These results are show that FLJ32028 has the topology of a Type Ia membrane protein.

### Example 3

### Antibody Production

To make monoclonal antibodies to the FLJ32028 extracellular domain (“ED”), mice were immunized with a purified FLJ32028(ED)-Fc fusion protein and/or with live 293-EBNA cells expressing full-length FLJ32028. Overlap extension PCR was used to construct a fusion gene containing amino acids 23-76 of the ED of FLJ32028 fused in frame to the Fc domain of mouse IgG1. The fusion gene was cloned into the baculovirus transfer vector pAcGP67-A (Pharmingen, San Diego, Calif. 92121). The vector contains the signal sequence from the baculovirus envelope protein gp67 for efficient secretion of the recombinant Fc fusion protein into the supernatant of infected cell cultures. This construct was used to produce recombinant baculovirus for expression and purification of the FLJ32028(ED)-Fc protein in insect cells. The protein was purified from 2 liters of baculovirus supernatant by FPLC on a goat anti-mouse IgG Fc fragment-specific affinity column.

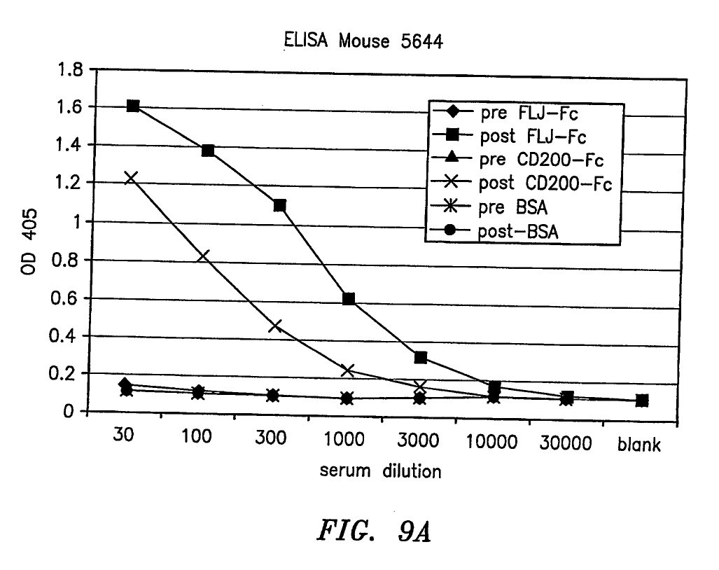

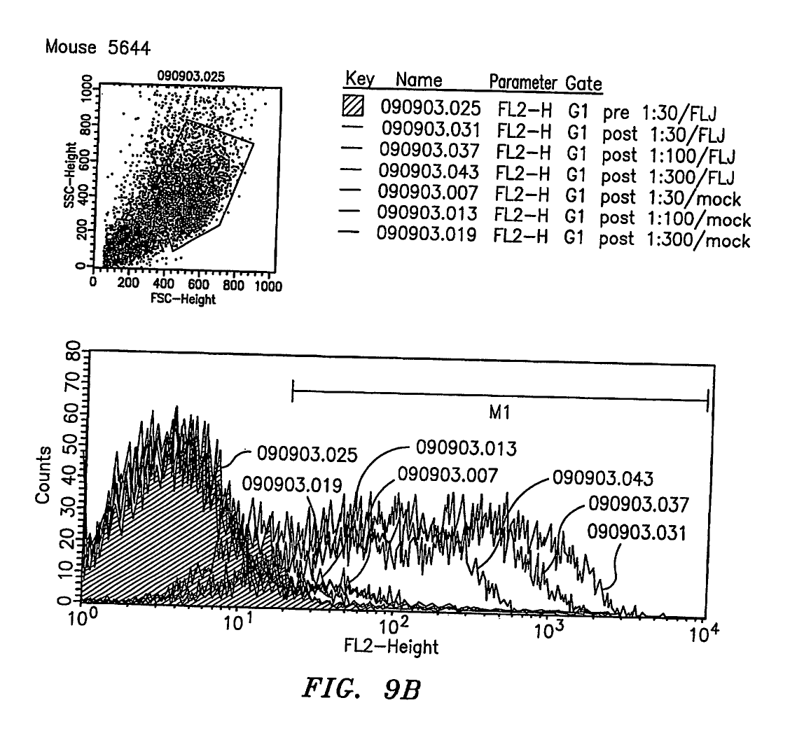

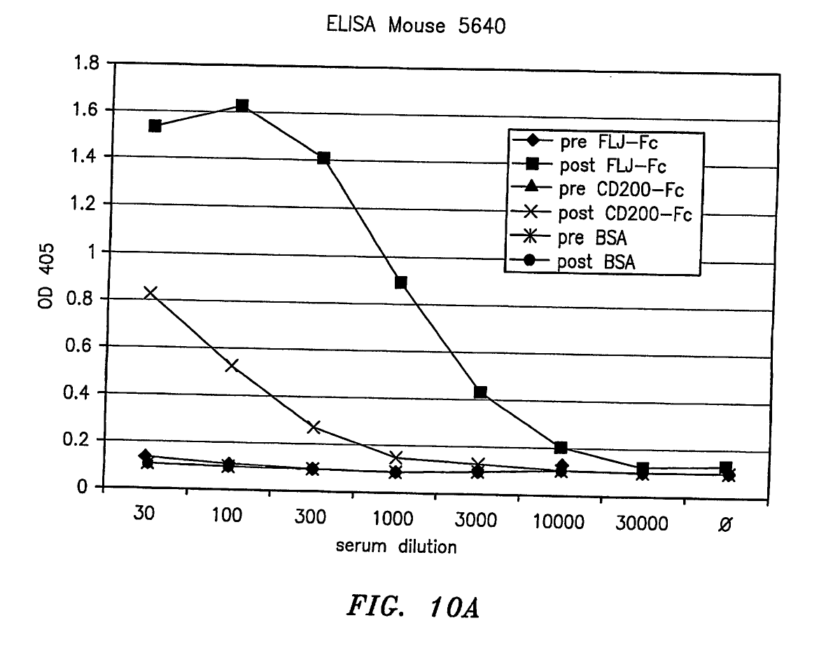

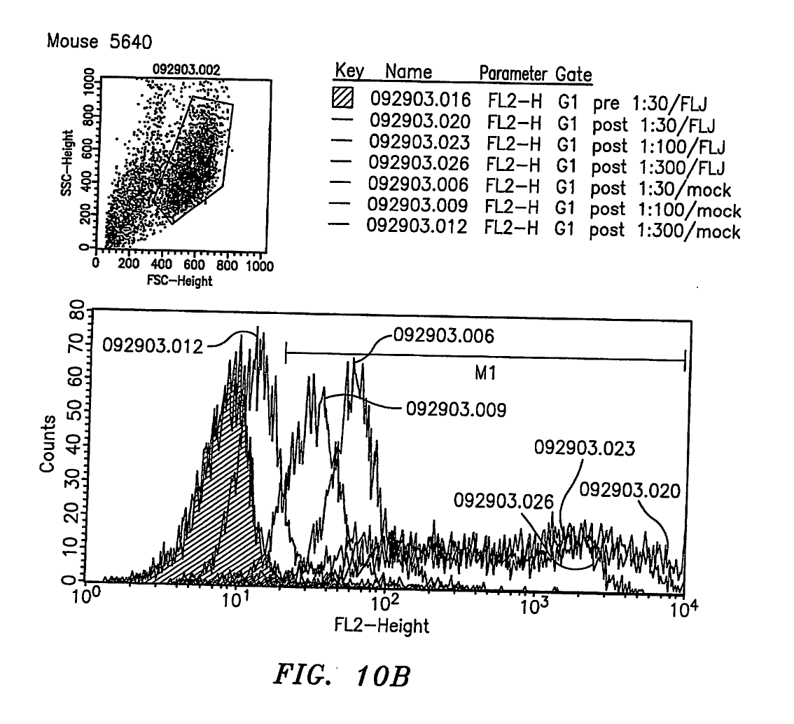

Six mice (5640-5645) were immunized with the FLJ32028(ED)-Fc protein or with a combination of the FLJ32028 (ED)-Fc protein and live 293-EBNA cells expressing full length FLJ32028 (293-FLJ cells) according to the schedule described in Table 2. Serum was collected on the day of the final boost and used to determine the antibody titers for the FLJ32028 ED (FIGS. 9 and 10). Serum titers were determined by ELISA on microtiter plates coated with FLJ32028(ED)-Fc and by flow cytometry on 293-FLJ cells. More specifically, Mouse 5644 was immunized with FLJ32028-Fc fusion protein as described in Table 2.

TABLE 2
Immunization schedule for mice.

| Mouse | 6/30/03# | 7/21/03# | 8/11/03# | 9/4/03* | 9/25/03* |
|---|---|---|---|---|---|
| 5640 | 293-FLJ | 293-FLJ | FLJ- | FLJ- | 293-FLJ |
| | | | Fc(25 μg) | Fc(25 μg) | |
| 5641 | 293-FLJ | 293-FLJ | FLJ- | FLJ- | 293-FLJ |
| | | | Fc(25 μg) | Fc(25 μg) | |
| 5643 | 293-FLJ | 293-FLJ | FLJ- | FLJ- | 293-FLJ |
| | | | Fc(25 μg) | Fc(25 μg) | |
| 5642 | FLJ- | FLJ- | FLJ- | FLJ- | — |
| | Fc(50 μg) | Fc(25 μg) | Fc(25 μg) | Fc(25 μg) | |
| 5644 | FLJ- | FLJ- | FLJ- | FLJ- | — |
| | Fc(50 μg) | Fc(25 μg) | Fc(25 μg) | Fc(25 μg) | |
| 5645 | FLJ- | FLJ- | FLJ- | FLJ- | — |
| | Fc(50 μg) | Fc(25 μg) | Fc(25 μg) | Fc(25 μg) | |

293-FLJ: 10⁶ 293-EBNA cells transfected with full length FLJ32028-HA gene (~90% transfection efficiency).
FLJ-Fc: Fusion protein containing the FLJ32028 extracellular domain and the Fc domain of mouse IgG1. ELJ-Fc was affinity purified from baculovirus supernatant using a Goat anti-Mouse Fc column.
#sub-cu injection at four sites
*sub-cu injection at four sites plus IV injection

On the day of the final boost, serum was isolated and the antibody titer was determined by ELISA (panel A) and flow cytometry (panel B). The ELISA protocol was as follows: microtiter wells were coated with 0.1 μg of FLJ32028-Fc protein (FLJ-Fc), CD200-Fc protein, or BSA. After blocking the wells with BSA, preimmune (re) or postimmune (post) serum (serially diluted from 30 to 30000-fold) was added for 1 hour at 37° C. Serum was removed by washing the wells 3 times with PBST. Bound antibodies were detected with an alkaline phosphatase-conjugated goat anti-mouse IgG F(ab′)₂ fragment-specific antibody (Jackson Immunoresearch, West Grove, Pa.) and PNPP substrate. Flow cytometry was conducted as follows: 293-EBNA cells were transiently transfected with the FLJ32028 cDNA in vector pCEP4 or with empty vector. Two days later, the cells were dissociated and labeled with postimmune serum at a dilution of 30, 100, or 300-fold or with preimmune serum at a dilution of 30-fold. Bound antibodies were detected using a PE-conjugated goat anti-mouse IgG antibody (Jackson Immunoresearch). The cells were analyzed on a BD FACSCalibur flow cytometer. Mouse 5640 was immunized with both FLJ32028-Fc fusion protein and 293-FLJ cells as described in Table 2. On the day of the final boost, serum was isolated and the antibody titer was determined by ELISA (panel A) and flow cytometry (panel B) as described hereinabove.

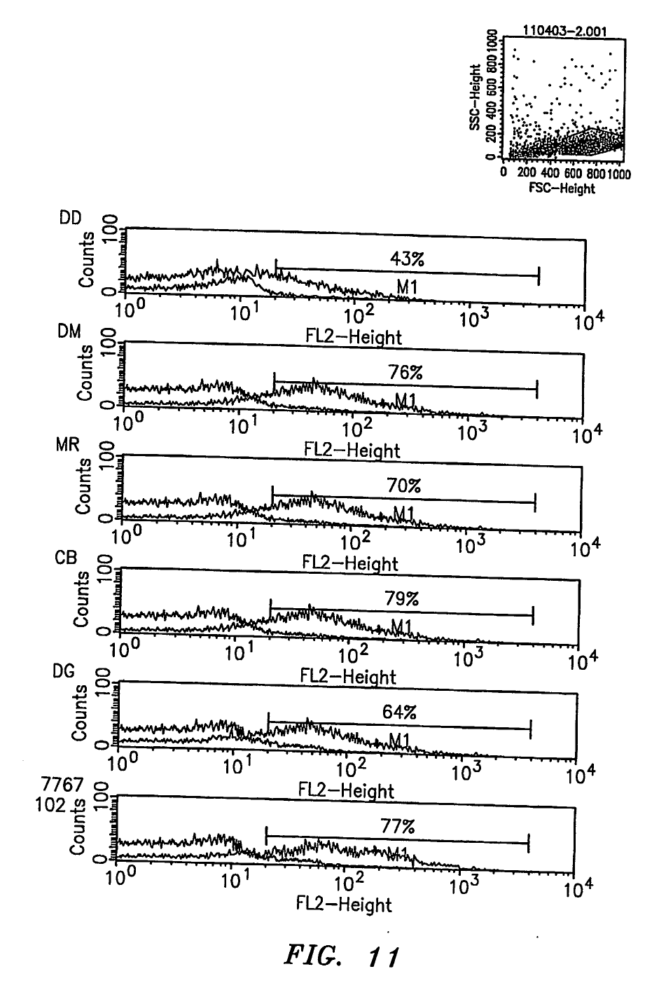

To determine if the antiserum recognized a cell surface antigen present on primary CLL cells, PBMC isolated from six patients presenting with CLL were stained with antiserum or preimmune serum from mouse 5644 and analyzed by flow cytometry (FIG. 11). More specifically, blood was collected from patients diagnosed with CLL and peripheral blood mononuclear cells (PBMCs) were isolated on a Histo-Paque gradient. The PBMCs were stained with preimmune or postimmune serum from mouse 5644 at a 25-fold dilution. Bound antibodies were detected using a PE-conjugated goat anti-mouse IgG antibody (Jackson Immunoresearch). The cells were analyzed on a BD FACSCalibur flow cytometer. The results showed that between 43-79% of the malignant cells from each patient expressed antigen recognized by the antiserum. Since CLL cells express high levels of the FLJ32028 mRNA, this antigen is most likely FLJ32028 protein.

Spleens were harvested from the mice and used to construct IgG1 and IgG2a Fab antibody phage display libraries as previously described in WO 03/025202A2, the disclosure of which is incorporated herein in its entirety. Libraries were constructed only from the two mice having the best antibody titers, 5644 and 5640.

The libraries made from mouse 5644 were panned on immobilized FLJ32028(ED)-Fc fusion protein to select antibodies to the FLJ 32028 ED. More specifically, poly-A RNA was isolated from the spleen of mouse 5644 and used to construct a Fab antibody phage display library. The library was subtracted with immobilized CD200-Fc protein to remove Fc-specific phage antibodies and then panned for three rounds on immobilized FLJ32028-Fc protein. 95 clones were picked after round 3 and analyzed by phage ELISA on microtiter plates coated with 0.1 μg of FLJ32028-Fc protein (R3 G1 FLJ) or CD200-Fc protein (R3 G1 CD). Bound phage were detected with an alkaline phosphatase-conjugated goat anti-mouse IgG F(ab′)₂ fragment-specific antibody (Jackson Immunoresearch) and PNPP substrate. Positive clones specific for the FLJ32028 extracellular domain are labeled above the bars. To determine the relative amounts of Fab produced by the clones (G1 Fab R3), microtiter plates were coated with a rabbit anti-mouse IgG F(ab′)₂ fragment-specific antibody. After blocking the wells with BSA, phage antibodies were added and incubated for 1 hour at 37° C. Unbound phage were removed by washing the wells 3 times with PBST. Bound phage antibodies were detected with an alkaline phosphatase-conjugated goat anti-mouse IgG F(ab′)₂ fragment-specific antibody (Jackson Immunoresearch) and PNPP substrate.

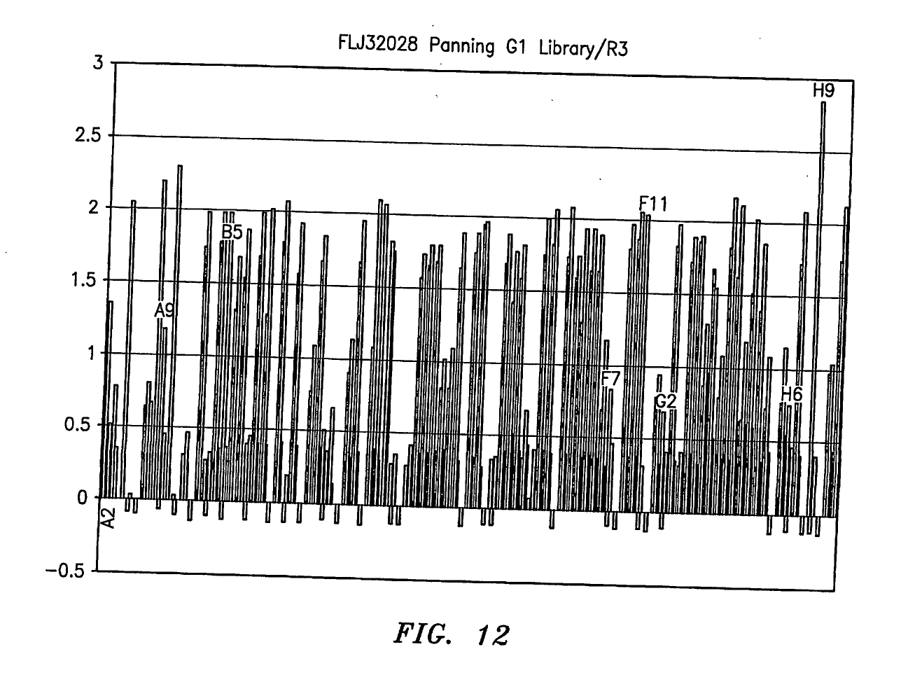

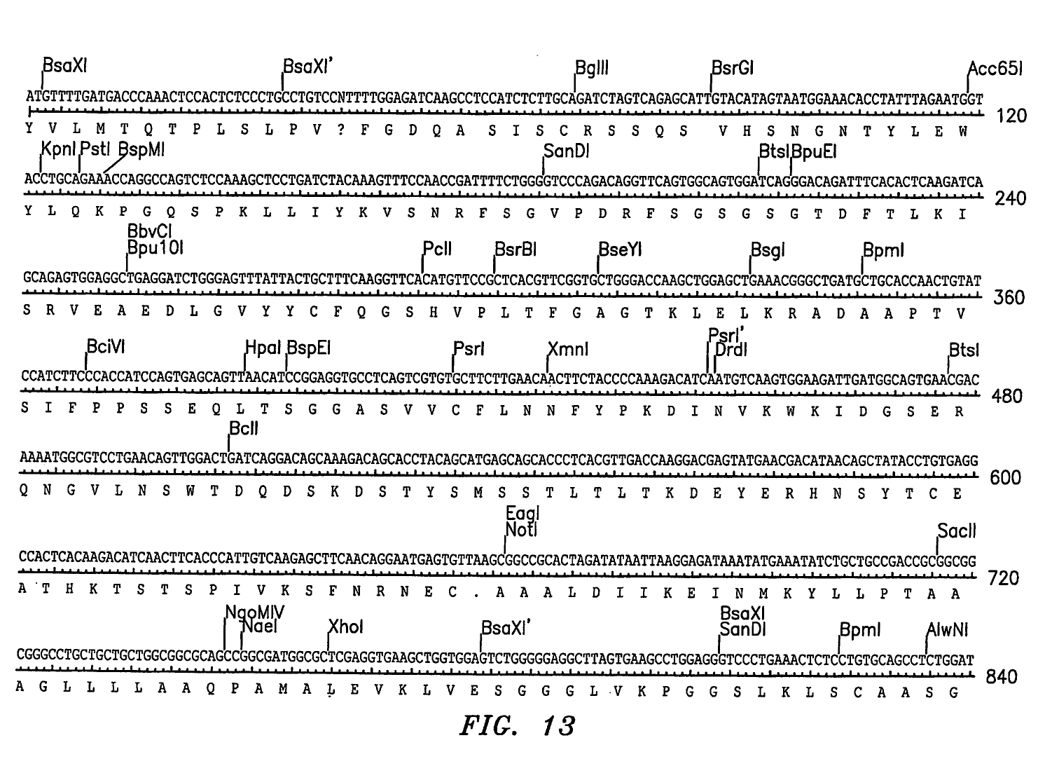

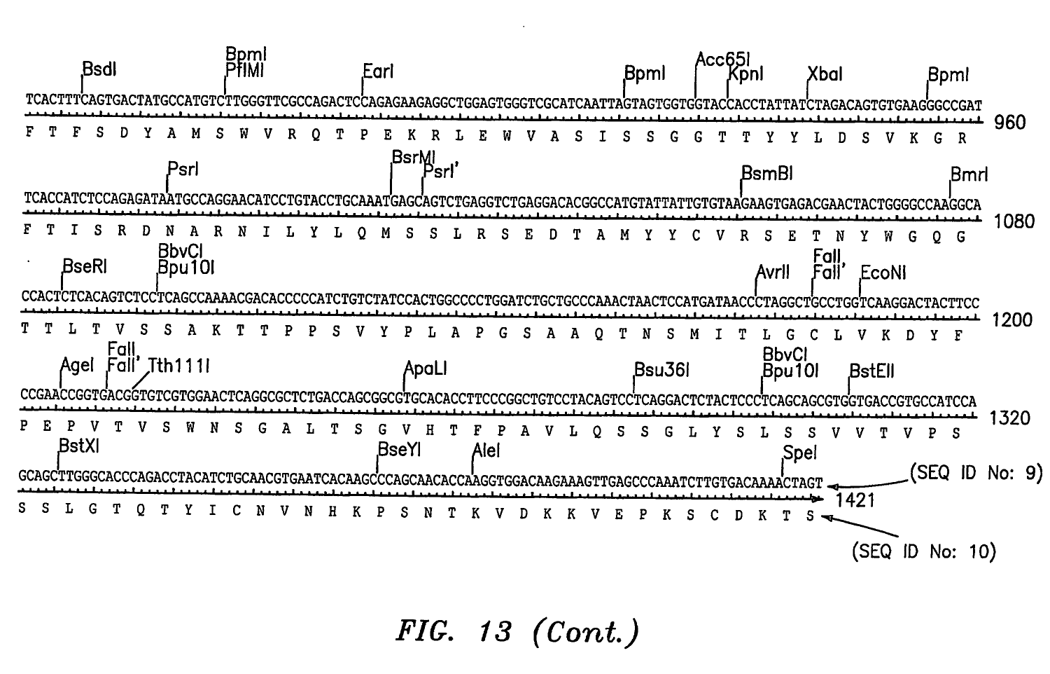

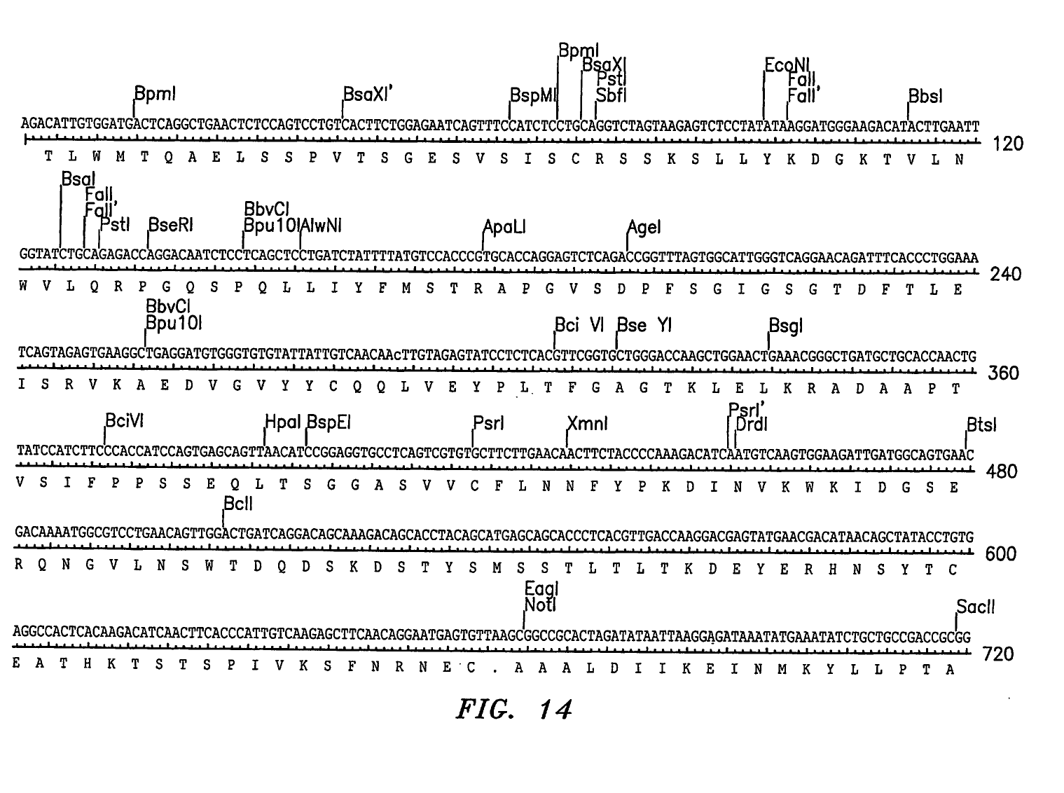

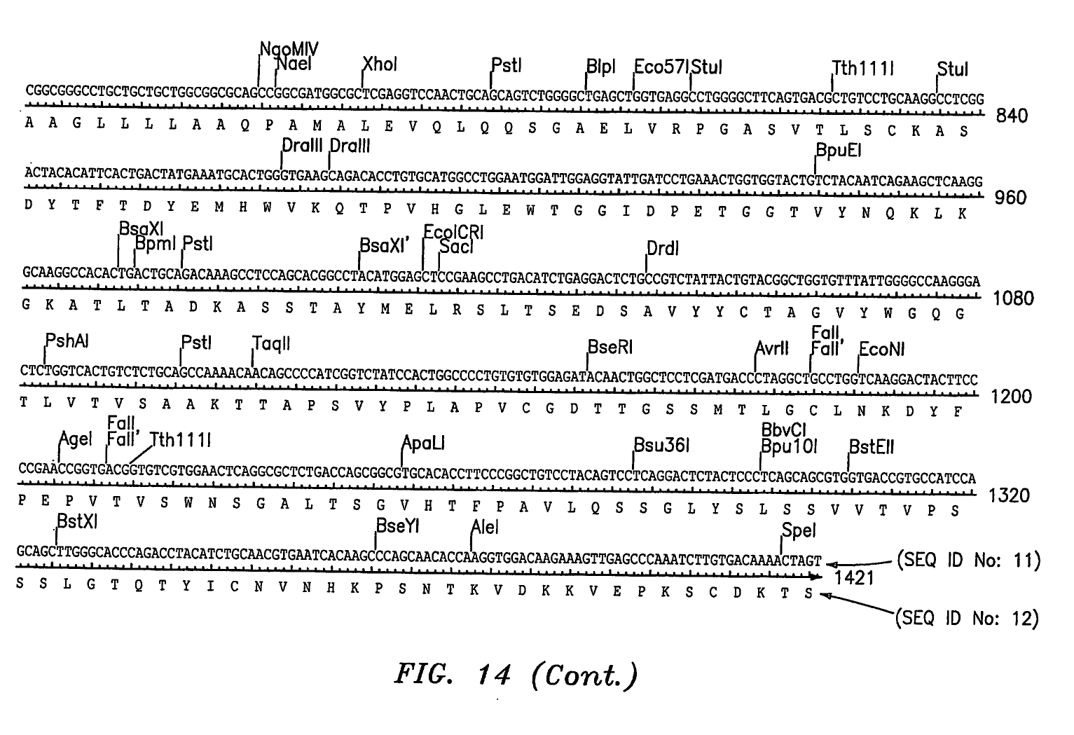

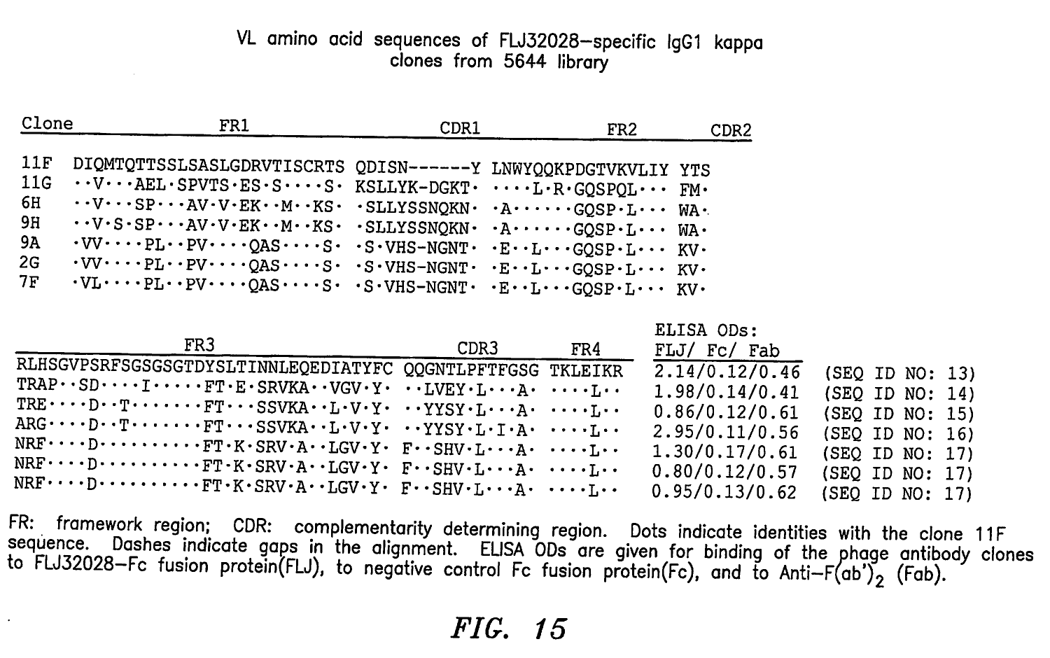

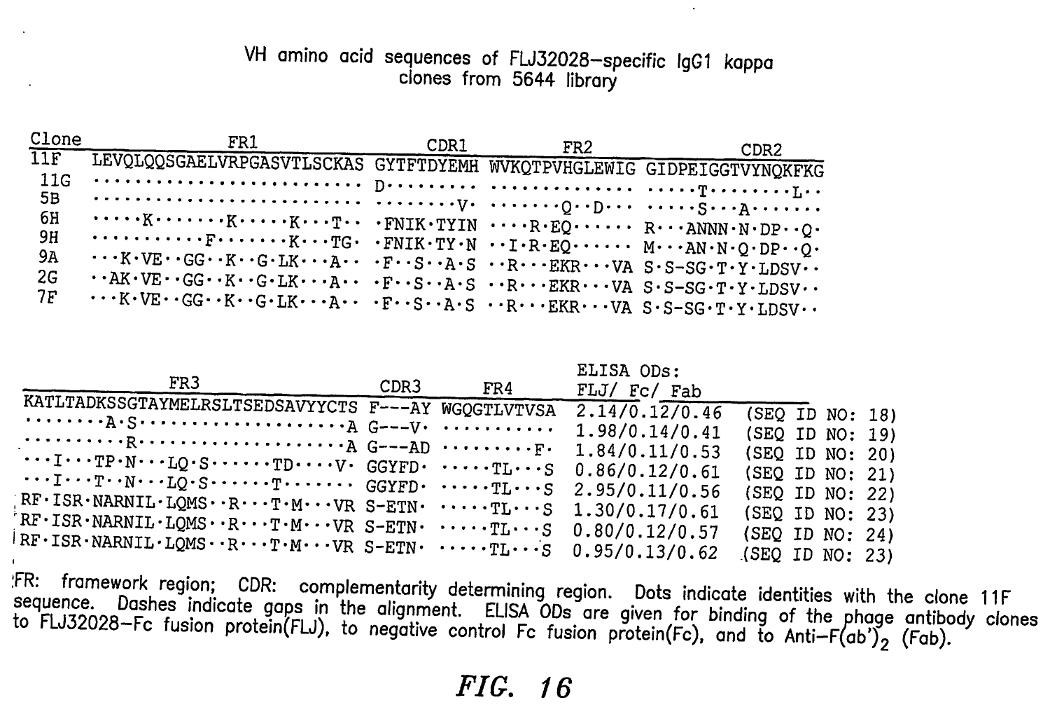

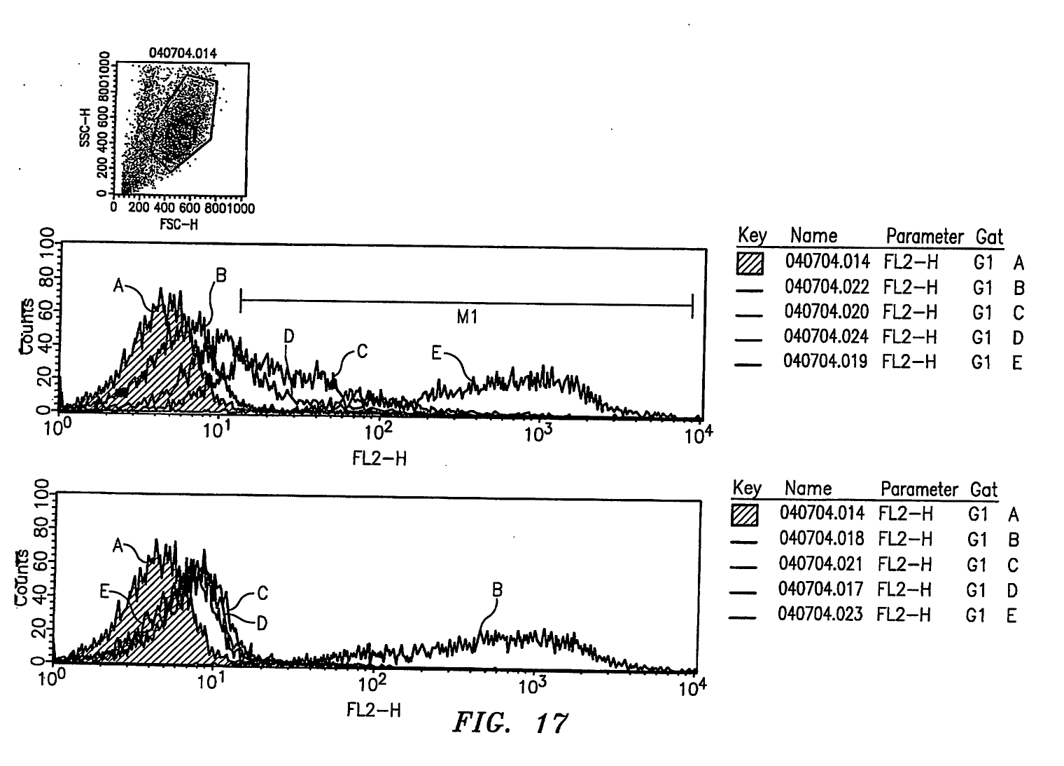

After the third round of panning, 95 clones from each library were screened by ELISA for specific binding to the FLJ32028 ED (FIG. 12). From the IgG1 library, 7 out of 95 clones bound to FLJ32028-Fc protein but not to the CD200-Fc protein used as a negative control (FIG. 12). From the IgG2a library, 1 out of 95 clones bound to FLJ32028-Fc but not to CD200-Fc (not shown). The DNA sequences of the positive clones are shown in FIGS. 13 and 14. FIGS. 15 and 16 show the variable region amino acid sequence alignments of FLJ32028-specific Fabs from the 5644 library. FIG. 17 shows the binding of FLJ32028-specific Fab antibodies from the 5644 library to 293-EBNA cells transiently transfected with FLJ32028. Periplasmic fractions were prepared from E. coli cultures(strain TOP10F′) expressing the Fab antibodies. The periplasmic fractions were incubated with 293-EBNA cells expressing the full length FLJ32028 cDNA. After washing the cells, bound Fabs were detected with a PE-conjugated Goat Anti-Mouse IgG, F(ab′)₂ fragment-specific secondary antibody. The cells were analysed using a FACSCalibur flow cytometer.

The libraries made from mouse 5640 were panned in the same way. FLJ32028 ED-specific antibodies selected from the four libraries were purified. Those antibodies with the highest affinity as determined by BIACORE assay were converted to mouse-human chimeric whole IgG format and expressed in mammalian cells. Cell-based assays were performed to test the ability of these chimeric antibodies to kill primary CLL cells or cell lines derived from CLL patients. Assays for cytotoxicity (e.g ATP-Lite), antibody-dependent cell-mediated cytotoxicity (ADCC), and complement-mediated cell lysis were performed.

The 5640 phage library was panned either on FLJ32028-Fc directly coated on to microtiter wells or captured with goat anti-mouse IgG Fc antibody. Many binders to either the Fc portion or the contaminating protein in the preparation were isolated and were used to masking epitopes for the next set. The antigen was pre-incubated with these Fabs prior to the addition of the library phage. Soluble CD200-Fc was also added to the phage suspension for the deselection of the binders to the Fc portion.

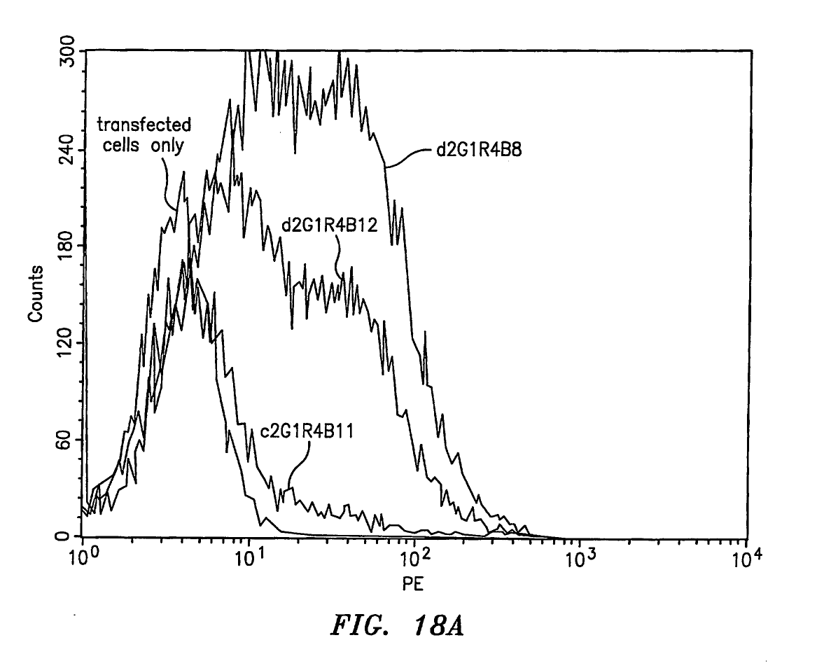

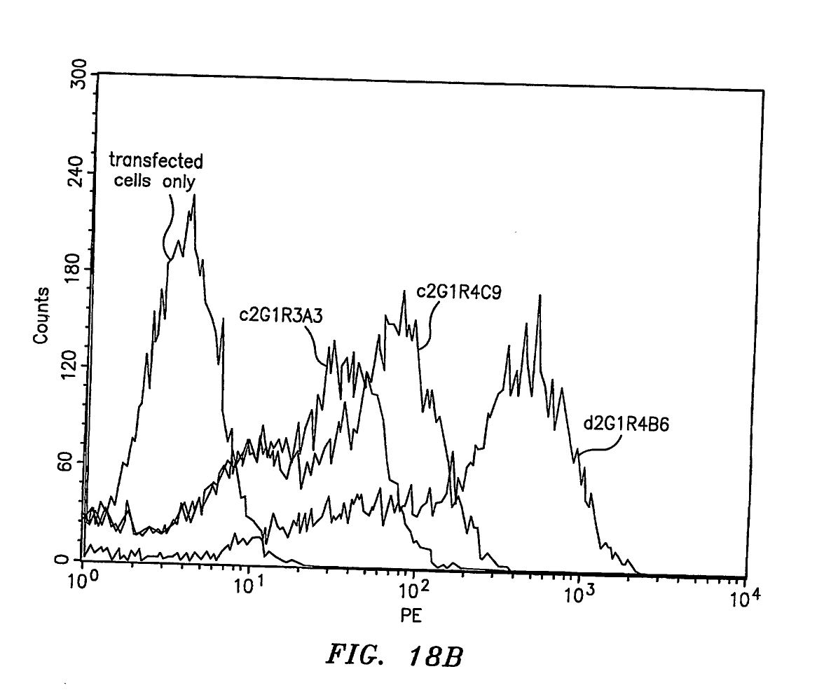

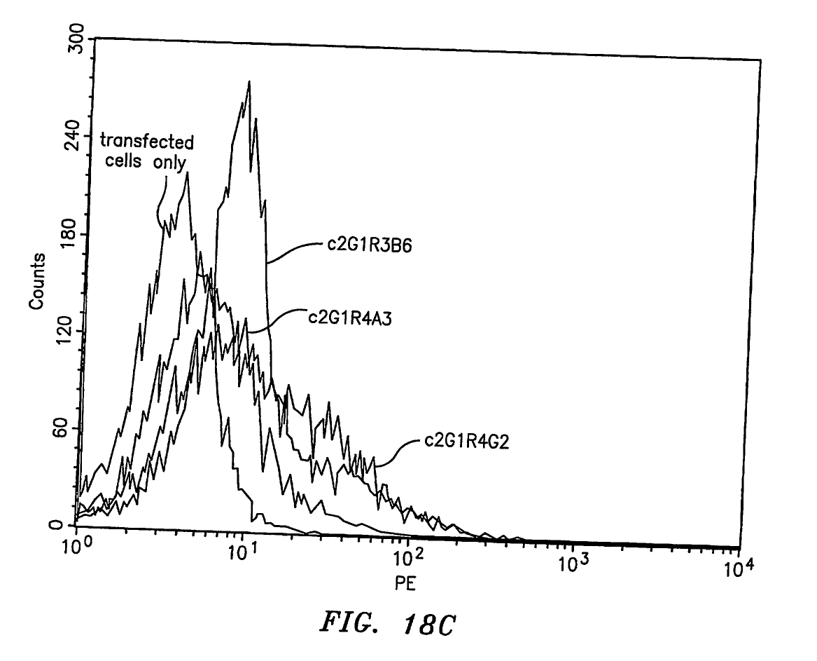

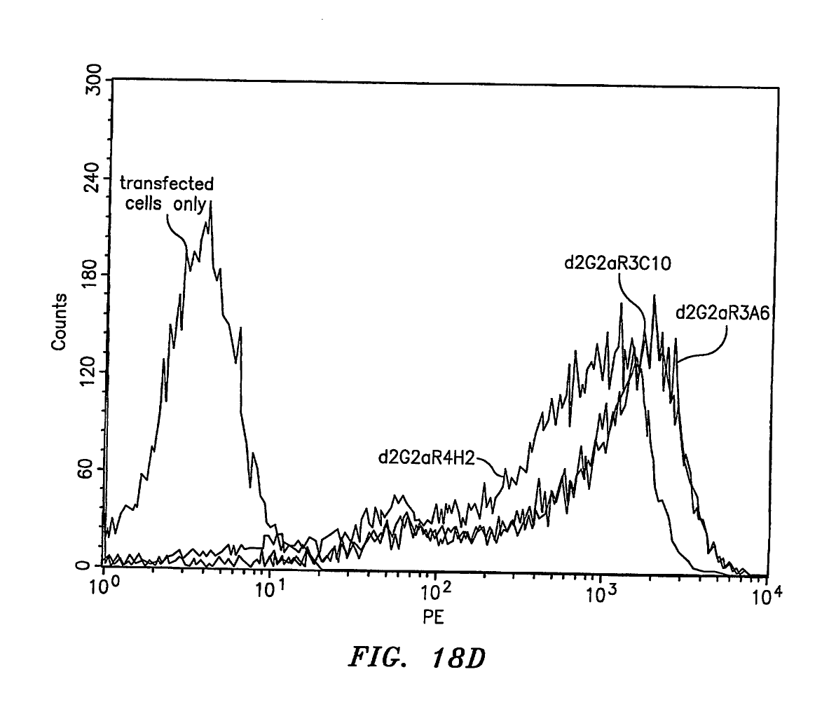

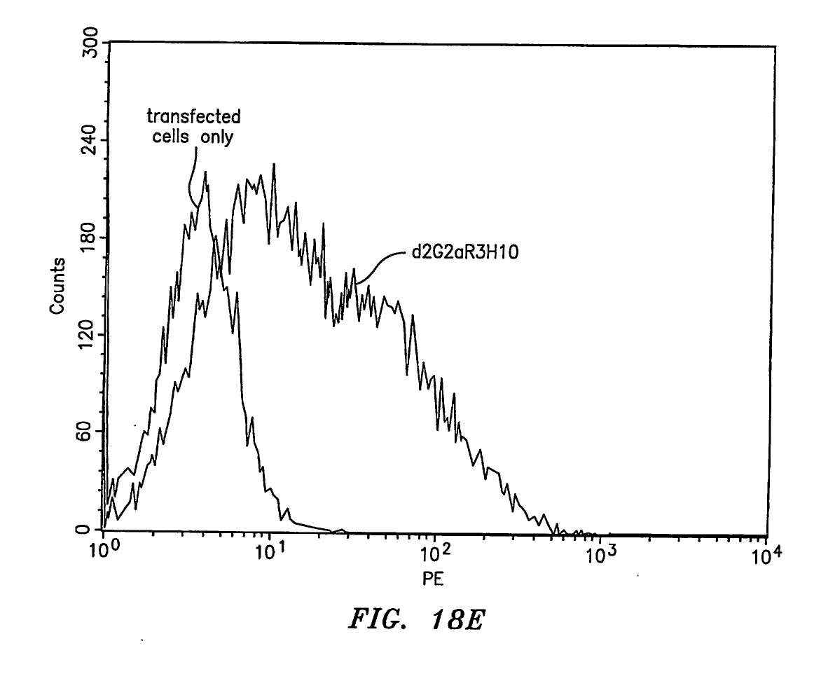

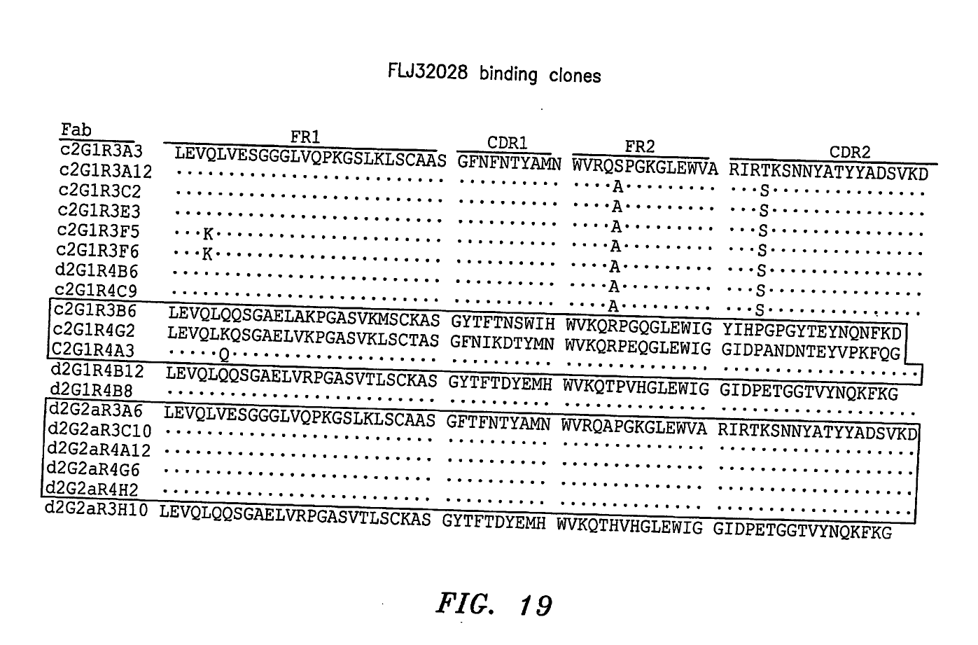

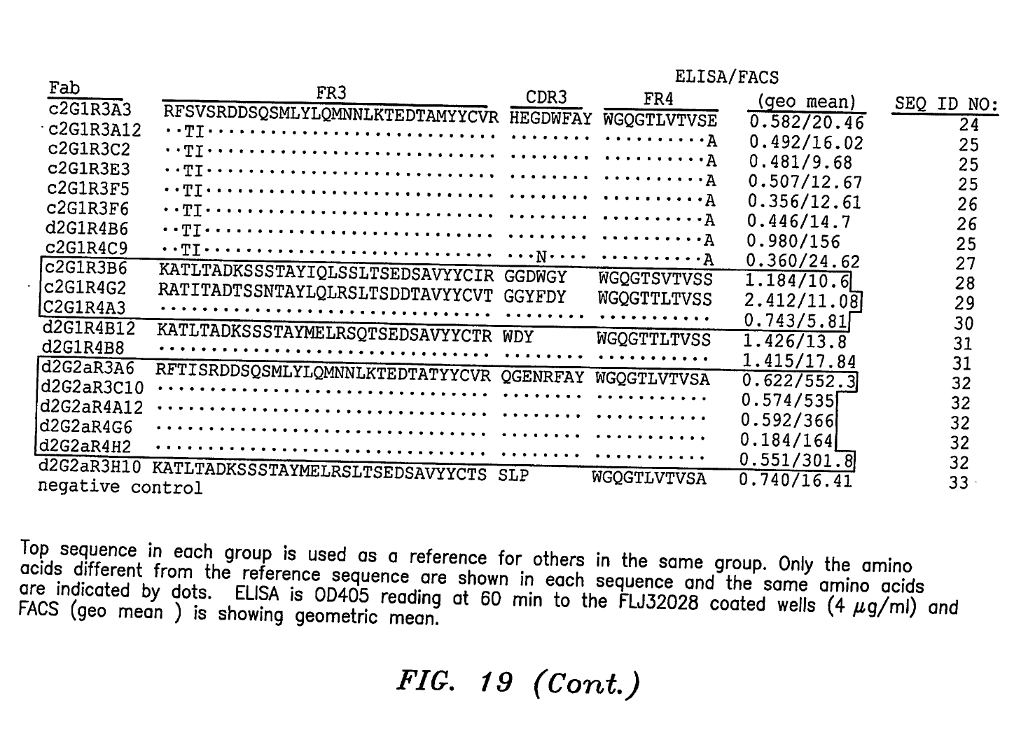

A large panel of FLJ32028-specific antibodies was isolated from IgG1 kappa and IgG2a kappa libraries. DNA sequences were analyzed and grouped according to the heavy chain complementarity-determining region 3 (HCDR3). Selected Fabs were tested by flow cytometry for the binding to FLJ32028 transfected 293 cells. FIGS. 18A-E show the flow cytometry results for some of the Fabs that showed binding to the FLJ32028 expressing cells. FIG. 19 shows the deduced amino acid sequences of the heavy chains of the clones that showed binding to FLJ32028 by flow cytometry.

It will be understood that various modifications may be made to the embodiments disclosed herein. Those skilled in the art will envision other modifications within the scope of the claims appended hereto.

1-16. (canceled)

17. An antibody which specifically binds to a polypeptide having at least 80% amino acid sequence identity to SEQ ID NO: 1.

18. The antibody of claim 17, wherein said antibody is a monoclonal antibody, a humanized antibody or a single-chain antibody.

19. The antibody of claim 17, wherein said antibody is a human antibody.

20. The antibody of claim 17, wherein said antibody is a murine antibody.

21. The antibody of claim 17, wherein the antibody comprises SEQ ID NO: 10.

22. The antibody of claim 17 wherein the antibody comprises SEQ ID NO: 12.

23. The antibody of claim 17 wherein the antibody comprises any of SEQ ID NOS: 13 through 33.

24. An isolated nucleic acid that encodes an antibody in accordance with claim 17.

25. The isolated nucleic acid of claim 24 wherein the isolated nucleic acid has at least 80% nucleic acid sequence identity to a nucleotide sequence shown of SEQ ID NO: 9.

26. The isolated nucleic acid of claim 24 wherein the isolated nucleic acid has at least 80% nucleic acid sequence identity to a nucleotide sequence shown of SEQ ID NO: 10.

27. A vector comprising the nucleic acid of claim 24.

28. A host cell comprising the vector of claim 27.

29. A process for producing an antibody comprising culturing the host cell of claim 28 under conditions suitable for expression of said antibody and recovering said antibody from the cell culture.

30. A method for identifying chronic lymphocytic leukemia cells, said method comprising contacting said cells with an anti-FLJ32028 antibody.

31. A method for detecting the presence of chronic lymphocytic leukemia cells in a mammal, said method comprising comparing the level of expression of FLJ32028 in (a) a test sample of cells taken from said mammal and (b) a control sample of normal cells of the same cell type, wherein a higher level of expression of said FLJ32028 in the test sample as compared to the control sample is indicative of the presence of chronic lymphocytic leukemia cells in said mammal.

32. (canceled)

33. An antibody in accordance with claim 17 that comprises a variable light chain CDR1 region that comprises an amino acid sequence selected from the group consisting of QDISNY (SEQ ID NO: 34), KSLLYKDGKTY (SEQ ID NO: 35), QSLLYSSNQKNY (SEQ ID NO: 36) and QSIVHSNGNTY (SEQ ID NO: 37).

34. An antibody in accordance with claim 17 that comprises a variable heavy chain CDR1 region that comprises an amino acid sequence selected from the group consisting of GYTFTDYEMH (SEQ ID NO:38), DYTFTDYEMH (SEQ ID NO: 39), GYTFTDYEVH (SEQ ID NO: 40), GFNIKDTYIN (SEQ ID NO: 41), GFTFSDYAMS (SEQ ID NO: 42), GFNFNTYAMN (SEQ ID NO: 43), GYTFTNSWIH (SEQ ID NO: 44), GFNIKDTYMN (SEQ ID NO: 45), GYTFTDYEMH (SEQ ID NO: 46), GFTFNTYAMN (SEQ ID NO: 47) and GYTFTDYEMH (SEQ ID NO:48).

35. An antibody in accordance with claim 17 that comprises a variable light chain CDR2 region that comprises an amino acid sequence selected from the group consisting of YTS (SEQ ID NO: 49), FMS (SEQ ID NO: 50), WAS (SEQ ID NO:51) and KVS (SEQ ID NO: 52).

36. An antibody in accordance with claim 17 that comprises a variable heavy chain CDR2 region that comprises an amino acid sequence selected from the group consisting of GIDPEIGGTVYNQKFKG (SEQ ID NO: 53), GIDPETGGTVYNQKLKG (SEQ ID NO: 54), GIDPESGGTAYNQKFKG (SEQ ID NO: 55), RIDPANNNTNYDPKFQG (SEQ ID NO: 56), MIDPANGNTQYDPKFQG (SEQ ID NO: 57), SISSGGTTYYLDSVKG (SEQ ID NO:58), RIRTKSNNYATYYADSVKD (SEQ ID NO: 59), RIRSKSNNYATYYADSVKD (SEQ ID NO: 60), YIHPGPGYTEYNQNFKD (SEQ ID NO:61), GIDPANDNTEYVPKFQG (SEQ ID NO: 62), GIDPETGGTVYNQKFKG (SEQ ID NO: 63), RIRTKSNNYATYYADSVKD (SEQ ID NO: 64) and GIDPETGGTVYNQKFKG (SEQ ID NO: 65).

37. An antibody in accordance with claim 17 that comprises a variable light chain CDR3 region that comprises an amino acid sequence selected from the group consisting of QQGNTLPFTFGSG (SEQ ID NO: 66), QQLVEYPLTFGAG (SEQ ID NO: 67), QQYYSYPLTFGAG (SEQ ID NO:68), QQYYSYPLTIGAG (SEQ ID NO: 69) and FQGSHVPLTFGAG (SEQ ID NO: 70).

38. An antibody in accordance with claim 17 that comprises a variable heavy chain CDR3 region that comprises an amino acid sequence selected from the group consisting of FAY (SEQ ID NO: 71), GVY (SEQ ID NO: 72), GAD (SEQ ID NO: 73), GGYFDY (SEQ ID NO: 74), SETNY (SEQ ID NO: 75), HEGDWFAY (SEQ ID NO: 76), HEGNWFAY (SEQ ID NO: 77), GGDWGY (SEQ ID NO: 78), GGYFDY (SEQ ID NO: 79), WDY (SEQ ID NO:80), QGENRFAY (SEQ ID NO:81) and SLP (SEQ ID NO:82).

39. An antibody comprising a heavy chain CDR1 region having the sequence GFTFNTYAMN (SEQ ID NO: 47).

40. An antibody comprising a heavy chain CDR2 region having the sequence RIRTKSNNYATYYADSVKD (SEQ ID NO: 59).

41. An antibody comprising a heavy chain CDR3 region having the sequence QGENRFAY (SEQ ID NO:81).

42. An antibody comprising a heavy chain CDR1 region having the sequence GFTFNTYAMN (SEQ ID NO: 47), a heavy chain CDR2 region having the sequence RIRTKSNNYATYYADSVKD (SEQ ID NO: 59) and a heavy chain CDR3 region having the sequence QGENRFAY (SEQ ID NO: 81).
